(19) United States
(12) Patent Application Publication
(10) Pub. No.: US 2026/0168030 A1
(43) Pub. Date: Jun. 18, 2026

(54) NUCLEIC ACID PROBE COMPOSITIONS FOR ENRICHING, EXTRACTING, AND/OR DETECTING A VARIANT TARGET NUCLEIC ACID IN A SAMPLE

(71) Applicant: GLC BIOTECHNOLOGY, INC., Hudson, OH (US)

(72) Inventors: Baochuan GUO, Hudson, OH (US); Qipan DENG, Hudson, OH (US)

(21) Appl. No.: 18/995,622

(22) PCT Filed: Aug. 15, 2023
(86) PCT No.: PCT/US2023/072192

§ 371 Date: Jan. 16, 2025

Related U.S. Application Data
(60) Provisional application No. 63/371,557, filed on Aug. 16, 2022.

(51) Int. Cl. C12Q 1/6886

(52) U.S. Cl. CPC C12Q 1/6886; C12Q 2600/156

(57) ABSTRACT

A nucleic acid probe composition for enriching, extracting, and/or detecting a variant target nucleic acid in a sample comprising the variant and a reference target nucleic acid is provided. The variant and reference each comprise a nucleic acid strand segment having a 5′ end, a 3′ end, and a nucleic acid sequence. The nucleic acid sequences of the strand segments are identical except that the variant comprises a substitution, insertion, or deletion of 1 to 100 nucleotides relative to the reference. The composition comprises a capture probe comprising an oligonucleotide that has 5′ and 3′ ends and comprises a variant target domain and either a 5′ upstream toehold domain or a 3′ downstream toehold domain. The composition also comprises an enhancement probe comprising an oligonucleotide that has 5′ and 3′ ends and comprises a reference target domain and either a 3′ downstream toehold domain or a 5′ upstream toehold domain.

### FIELD OF THE INVENTION

The invention relates to nucleic acid probe compositions for enriching, extracting, and detecting a variant target nucleic acid in a sample, and more particularly to such nucleic acid probe compositions comprising a capture probe and an enhancement probe.

### BACKGROUND

Variations in nucleic acid sequences, including DNA and RNA, play important roles in many diseases. Detection of nucleic acid sequence variants at base-pair resolution is important for diagnosis and treatment. Many technologies have been developed for accomplishing such detection, including hybridization, polymerase chain reaction (“PCR”), isothermal amplification, and sequencing, individually and in combination. While each of these technologies offer advantages, each also includes drawbacks. For example, PCR-based detection methods often result in false positives and artificial mutations due to replication errors (slippage), and sequencing exhibits an intrinsic error rate of up to about 1.0% per nucleotide. This can make it challenging to detect sequence variants such as single nucleotide variants, particularly rare mutations.

Competitive hybridization reactions can be used for detection of nucleic acid sequence variants. See, e.g., Jeffreys et al., Genome Research 13:2316-2324 (2003). For example, Jeffreys et al. (2003) has reported a method for enriching mutated DNA in which the presence of competing probes that are specific to wild-type alleles could improve enrichment of the mutated DNA.

Toehold-mediated strand displacement (“toehold displacement”) and toehold exchange are competitive hybridization reactions that have been applied in various ways for detection of nucleic acid sequence variants. See, e.g., Zhang et al., Science 318:1121-1125 (2007); Zhang et al., Journal of the American Chemical Society 131:17303-17314 (2009); Zhang et al., Nature Chemistry 4:208-214 (2012); Yu et al., RSC Advances 7:40858-40865 (2017).

Toehold displacement involves an incoming nucleic acid strand outcompeting and thereby displacing an incumbent strand of a nucleic acid duplex to form a better matched duplex with a complementary remaining strand of the duplex. See, e.g., Yurke et al., Nature 406:605-608 (2000); Yu et al. (2017). This is accomplished based on the incoming strand having a unique toehold domain that includes about 4 to 10 nucleotides, is complementary to the remaining strand, and is not present in the incumbent strand. The toehold domain of the incoming strand binds to the remaining strand by the toehold, forming an intermediate complex composed of three strands. Branch migration of the invading strand then allows displacement of the incumbent strand, resulting in formation of the better matched duplex.

Toehold displacement is controlled by the Gibbs free energy of hybridization. The initial step of the incoming strand binding the remaining strand by the toehold is endothermic and rate limiting. The step can be tuned over a range of six orders of magnitude by varying the strength of the toehold domain, for example based on varying its length and/or G/C content. This allows kinetic control. The overall process is energetically favored. Although a reverse reaction can occur, its rate is up to six orders of magnitude slower.

Toehold exchange is similar to toehold displacement with respect to having an incoming nucleic acid strand and an incumbent strand, but differs in that the incoming strand and the incumbent strand each have a unique toehold domain that is absent from the other strand. Completion of toehold exchange thus requires binding of the incoming strand to the remaining strand by its unique toehold, and spontaneous dissociation of the incumbent strand from the remaining strand at its unique toehold. Toehold exchange possesses fast reaction kinetics and high specificity.

Toehold displacement probes and toehold exchange probes have been applied in various methods for detecting target nucleic acids, including variant target nucleic acids, that rely on specificity of nucleic acid hybridization.

For example, toehold displacement probes have been used to capture target DNAs in samples on magnetic beads. See, e.g., Fenati et al., International Journal of Nanotechnology 14:75-86 (2017); Kennedy-Darling et al., Chembiochem 15:2353-2356 (2014). In accordance with these methods, a double stranded toehold displacement probe is premade and pre-immobilized on a surface of a solid such as magnetic beads. Target DNA in a sample is contacted by the toehold displacement probe. The target DNA replaces one of the strands of the double stranded toehold displacement probe. The target DNA is thus captured by the magnetic beads through the toehold displacement.

Also for example, toehold exchange probes have been used to detect the presence of variant target DNAs in samples. See, e.g., Yu et al. (2017); Zhou et al., Langmuir 34:14811-14816 (2018); Wu et al., Biosensors and Bioelectronics 80:175-181 (2016). In accordance with these methods, a double stranded probe is made including a toehold exchange probe strand. A variant target nucleic acid in a sample is contacted with the double stranded probe to initiate strand displacement. Release of a detection probe strand from the double stranded probe is monitored as an indicator of the presence of the variant target nucleic acid in the sample.

There is a need for additional methods for detecting variant target nucleic acids.

### BRIEF SUMMARY OF THE INVENTION

A nucleic acid probe composition for enriching, extracting, and detecting a variant target nucleic acid in a sample comprising the variant target nucleic acid and a reference target nucleic acid is disclosed. The variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of one to 100 nucleotides, or a combination thereof, relative to the nucleic acid sequence of the nucleic acid strand segment of the reference target nucleic acid.

The composition comprises:
(a) a capture probe comprising a single-stranded oligonucleotide that has a 5′ end and a 3′ end and comprises a variant target domain and either a 5′ upstream toehold domain or a 3′ downstream toehold domain, but not both, contiguous to the variant target domain; and
(b) an enhancement probe comprising a single-stranded oligonucleotide that has a 5′ end and a 3′ end and comprises a reference target domain and either a 3′ downstream toehold domain or a 5′ upstream toehold domain, but not both, contiguous to the reference target domain.

The 5′ upstream toehold domain has a nucleic acid sequence that is a reverse complement of 3′ subsequences of the nucleic acid sequences of the strand segments of the variant and reference target nucleic acids extending from the 3′ ends of the strand segments of the variant and reference target nucleic acids.

The 3′ downstream toehold domain has a nucleic acid sequence that is a reverse complement of 5′ subsequences of the nucleic acid sequences of the strand segments of the variant and reference target nucleic acids extending from the 5′ ends of the strand segments of the variant and reference target nucleic acids.

The variant target domain has a nucleic acid sequence that is a reverse complement of an intermediate subsequence of the strand segment of the variant target nucleic acid that extends contiguously between the 5′ and 3′ subsequences of the strand segment of the variant target nucleic acid and comprises the substitution, insertion, or deletion, or combination thereof.

The reference target domain has a nucleic acid sequence that is a reverse complement of an intermediate subsequence of the strand segment of the reference target nucleic acid that extends contiguously between the 5′ and 3′ subsequences of the strand segment of the reference target nucleic acid and does not comprise the substitution, insertion, or deletion, or combination thereof.

If the capture probe comprises the 5′ upstream toehold domain, then the enhancement probe comprises the 3′ downstream toehold domain, whereas if the capture probe comprises the 3′ downstream toehold domain, then the enhancement probe comprises the 5′ upstream toehold domain.

The nucleic acid sequences of the 5′ upstream toehold domain and the 3′ downstream toehold domain each comprise 2 to 100 nucleotides.

The composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

In some embodiments, the single-stranded oligonucleotide of the capture probe comprises one or more of DNA, RNA, locked nucleic acids, or peptide nucleic acids, or a combination thereof; and the single-stranded oligonucleotide of the enhancement probe comprises one or more of DNA, RNA, locked nucleic acids, or peptide nucleic acids, or a combination thereof.

In some embodiments, the substitution, insertion, or deletion, or combination thereof, comprises one or more of a single-base substitution, a multiple-base substitution, a single-base insertion, a multiple-base insertion, a single-base deletion, or a multiple-base deletion.

In some embodiments, the composition further comprises a guanidine-based salt. In some of these embodiments, the guanidine-based salt comprises one or more of guanidine or a guanidine derivative with the structure (R1R2N)(R3R4N)C═N—R5, wherein R1, R2, R3, R4, and R5 each independently are either (i) hydrogen or (ii) a functional group comprising 1 to 4 carbons with hydrogens, with the proviso that the sum of carbons in R1 to R5 is less than or equal to 15. Also in some of these embodiments, the composition is aqueous and the guanidine-based salt is present in the composition at a concentration of 0.05 to 8 M.

In some embodiments, the nucleic acid sequences of the strand segments of the variant and reference target nucleic acids each comprise 15 to 400 nucleotides.

In some embodiments, the variant target domain and the reference target domain each comprise 1 to 200 nucleotides.

In some embodiments, the capture probe comprises only one capture probe and the enhancement probe comprises only one enhancement probe, wherein the capture probe is specific to a single variant target nucleic acid, and the enhancement probe is specific to a single reference target nucleic acid.

In some embodiments, the capture probe comprises a plurality of capture probes, and the enhancement probe comprises only one enhancement probe, wherein each of the capture probes is specific to a different variant target nucleic acid, and the enhancement probe is specific to a single reference target nucleic acid.

In some embodiments, the capture probe comprises a plurality of capture probes, and the enhancement probe comprises a plurality of enhancement probes, wherein each of the capture probes is specific to a different variant target nucleic acid, and each of the enhancement probes is specific to a different reference target nucleic acid, wherein each of the reference target nucleic acids is a reference nucleic acid to one or more of the variant target nucleic acids.

In some embodiments, the composition does not comprise the variant or reference target nucleic acids.

In some embodiments, the composition comprises one or more of the variant and/or reference target nucleic acids.

In some embodiments, the capture probe comprises at least 6 pairs of adjacent matched nucleotides with respect to the nucleic acid sequence of the strand segment of the variant target nucleic acid, and the enhancement probe comprises at least 6 pairs of adjacent matched nucleotides with respect to the nucleic acid sequence of the strand segment of the reference target nucleic acid.

In some embodiments, the capture probe contains only matched nucleotides with respect to the nucleic acid sequence of the strand segment of the variant target nucleic acid, and the enhancement probe contains only matched nucleotides with respect to the nucleic acid sequence of the strand segment of the reference target nucleic acid.

In some embodiments, the composition does not comprise any other oligonucleotides that are complementary to the 5′ subsequences, the intermediate subsequences, or the 3′ subsequences of the strand segments of the variant or reference target nucleic acids and does not comprise any other nucleic acids that are complementary to the capture probe or the enhancement probe.

A system for enriching, extracting, and detecting a variant target nucleic acid in a sample comprising the variant target nucleic acid and a reference target nucleic acid is also disclosed. In the system, the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid.

The system comprises:
(a) the composition as described above; and
(b) a solid, wherein the capture probe is immobilized on a surface of the solid.

A system for enriching, extracting, and detecting a variant target nucleic acid in a sample comprising the variant target nucleic acid and a reference target nucleic acid also is disclosed. In the system, the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid.

The system comprises:
(a) the composition as described above; and
(b) a solid.

The capture probe comprises at least one binding tag.

The solid comprises an extraction tag that binds to the at least one binding tag.

A method for enriching, extracting, and detecting a variant target nucleic acid in a sample comprising the variant target nucleic acid and a reference target nucleic acid also is disclosed. The variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid.

The method comprises steps of:
(1) combining the sample, a capture probe, and an enhancement probe, thereby forming a hybridization composition;
(2) incubating the hybridization composition at a temperature that promotes the formation of hybrids of the capture probe and the variant target nucleic acid and hybrids of the enhancement probe and the reference target nucleic acid by toehold displacement and/or toehold exchange;
(3) isolating the hybrids formed by the capture probe from the hybrids formed by the enhancement probe;
(4) eluting the variant target nucleic acid from the hybrids of the capture probe and the variant target nucleic acid; and
(5) detecting the variant target nucleic acid.

The capture probe comprises a single-stranded oligonucleotide that has a 5′ end and a 3′ end and comprises a variant target domain and either a 5′ upstream toehold domain or a 3′ downstream toehold domain, but not both, contiguous to the variant target domain.

The enhancement probe comprises a single-stranded oligonucleotide that has a 5′ end and a 3′ end and comprises a reference target domain and either a 3′ downstream toehold domain or a 5′ upstream toehold domain, but not both, contiguous to the reference target domain.

The 5′ upstream toehold domain has a nucleic acid sequence that is a reverse complement of 3′ subsequences of the nucleic acid sequences of the strand segments of the variant and reference target nucleic acids extending from the 3′ ends of the strand segments of the variant and reference target nucleic acids.

The 3′ downstream toehold domain has a nucleic acid sequence that is a reverse complement of 5′ subsequences of the nucleic acid sequences of the strand segments of the variant and reference target nucleic acids extending from the 5′ ends of the strand segments of the variant and reference target nucleic acids.

The variant target domain has a nucleic acid sequence that is a reverse complement of an intermediate subsequence of the strand segment of the variant target nucleic acid that extends contiguously between the 5′ and 3′ subsequences of the strand segment of the variant target nucleic acid and comprises the substitution, insertion, or deletion, or combination thereof.

The reference target domain has a nucleic acid sequence that is a reverse complement of an intermediate subsequence of the strand segment of the reference target nucleic acid that extends contiguously between the 5′ and 3′ subsequences of the strand segment of the reference target nucleic acid and does not comprise the substitution, insertion, or deletion, or combination thereof.

If the capture probe comprises the 5′ upstream toehold domain, then the enhancement probe comprises the 3′ downstream toehold domain, whereas if the capture probe comprises the 3′ downstream toehold domain, then the enhancement probe comprises the 5′ upstream toehold domain.

The nucleic acid sequences of the 5′ upstream toehold domain and the 3′ downstream toehold domain each comprise 2 to 100 nucleotides.

The hybridization composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

In some embodiments, the method further comprises a step (0) of combining the sample and the capture probe, thereby forming an initial sample-capture probe composition prior to step (1).

In some embodiments, the method further comprises a step (0) of combining the sample and the enhancement probe, thereby forming an initial sample-enhancement probe composition prior to step (1).

In some embodiments, (i) the method further comprises a step (0) of immobilizing the capture probe on a surface of a solid prior to step (1); and (ii) step (3) comprises isolating the hybrids formed by the capture probe from the hybrids formed by the enhancement probe by separating the solid from the hybridization composition.

In some embodiments, (i) the capture probe comprises at least one binding tag; and (ii) step (3) comprises isolating the hybrids formed by the capture probe from the hybrids formed by the enhancement probe by contacting the hybridization solution with a solid that comprises an extraction tag that binds to the at least one binding tag, then separating the solid from the hybridization composition.

In some embodiments, step (5) comprises detecting the variant target nucleic acid by a DNA analysis method comprising one or more of real-time PCR, microarray, fragment size analysis, Sanger sequencing, next-generation sequencing, or digital PCR, or a combination thereof.

In some embodiments, the oligonucleotide of the capture probe comprises one or more of DNA, RNA, locked nucleic acids, or peptide nucleic acids, or a combination thereof; and the oligonucleotide of the enhancement probe comprises one or more of DNA, RNA, locked nucleic acids, or peptide nucleic acids, or a combination thereof.

In some embodiments, the substitution, insertion, or deletion comprises one or more of a single-base substitution, a multiple-base substitution, a single-base insertion, a multiple-base insertion, a single-base deletion, or a multiple-base deletion.

In some embodiments, the hybridization solution further comprises a guanidine-based salt. In some of these embodiments, the guanidine-based salt comprises one or more of guanidine or a guanidine derivative with the structure (R1R2N)(R3R4N)C═N—R5, wherein R1, R2, R3, R4, and R5 each independently are either (i) hydrogen or (ii) a functional group comprising 1 to 4 carbons with hydrogens, with the proviso that the sum of carbons in R1 to R5 is less than or equal to 15. Also in some of these embodiments, the guanidine-based salt is present in the hybridization composition at a concentration of 0.05 to 8 M.

In some embodiments, the nucleic acid sequences of the strand segments of the variant and reference target nucleic acids each comprise 15 to 400 nucleotides.

In some embodiments, the variant target domain and the reference target domain each comprise 1 to 200 nucleotides.

In some embodiments, the capture probe comprises only one capture probe and the enhancement probe comprises only one enhancement probe, wherein the capture probe is specific to a single variant target nucleic acid, and the enhancement probe is specific to a single reference target nucleic acid.

In some embodiments, the capture probe comprises a plurality of capture probes, and the enhancement probe comprises only one enhancement probe, wherein each of the capture probes is specific to a different variant target nucleic acid, and the enhancement probe is specific to a single reference target nucleic acid.

In some embodiments, the capture probe comprises a plurality of capture probes, and the enhancement probe comprises a plurality of enhancement probes, wherein each of the capture probes is specific to a different variant target nucleic acid, and each of the enhancement probes is specific to a different reference target nucleic acid, wherein each of the reference target nucleic acids is a reference nucleic acid to one or more of the variant target nucleic acids.

In some examples, the capture probe comprises at least 6 pairs of adjacent matched nucleotides with respect to the nucleic acid sequence of the strand segment of the variant target nucleic acid, and the enhancement probe comprises at least 6 pairs of adjacent matched nucleotides with respect to the nucleic acid sequence of the strand segment of the reference target nucleic acid.

In some examples, the capture probe contains only matched nucleotides with respect to the nucleic acid sequence of the strand segment of the variant target nucleic acid, and the enhancement probe contains only matched nucleotides with respect to the nucleic acid sequence of the strand segment of the reference target nucleic acid.

In some embodiments, the hybridization composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

### BRIEF DESCRIPTION OF THE DRAWINGS

These and other features, aspects, and advantages of the present disclosure are better understood when the following detailed description is read with reference to the accompanying drawings.

FIG. 1 shows a schematic diagram of variant and target sequences for a capture probe including a 5′ upstream toehold domain and an enhancement probe including a 3′ downstream toehold domain.

FIG. 2 shows a schematic diagram of variant and target sequences for a capture probe including a 3′ downstream toehold domain and an enhancement probe comprising a 5′ upstream toehold domain.

FIG. 3 shows example sequences of a capture probe, variant target nucleic acid, enhancement probe, and reference target nucleic acid. In FIG. 3(A), the capture probe (SEQ ID NO: 25) hybridizes preferentially with the variant target nucleic acid (SEQ ID NO: 26) and includes a 3′ downstream toehold domain, whereas the enhancement probe (SEQ ID NO: 27) hybridizes preferentially with the reference target nucleic acid (SEQ ID NO: 28) and includes a 5′ upstream toehold domain. In FIG. 3(B), the capture probe (SEQ ID NO: 29) hybridizes preferentially with the variant target nucleic acid (SEQ ID NO: 26) and includes a 5′ upstream toehold domain, whereas the enhancement probe (SEQ ID NO: 30) hybridizes preferentially with the reference target nucleic acid (SEQ ID NO: 28) and includes a 3′ downstream toehold domain. In both FIG. 3(A) and FIG. 3(B), the portions of the capture probes and enhancement probes corresponding to 5′ upstream toehold domains or 3′ downstream toehold domains are shown in italics, the portions of the variant and reference target nucleic acids that are the strand segments of the variant and reference target nucleic acids, respectively, are shown underlined, the portions of the intermediate subsequences that are identical between the strand segments of the variant and reference target nucleic acids are shown in underlined, and the portions of the intermediate subsequences that differ between the strand segments of the variant and reference target nucleic acids are shown in bold.

FIG. 4 shows a schematic diagram of hybridization in the method for enriching, extracting, and detecting variant target nucleic acids in a sample comprising variant and reference target nucleic acids. The method includes contacting a sample comprising variant and reference target nucleic acids with the nucleic acid probe composition comprising a capture probe and an enhancement probe for hybridization, in which some capture probes bind to variant target nucleic acids, while others bind to reference target nucleic acids. In the same way, some enhancement probes bind to variant target nucleic acids, while others bind to reference target nucleic acids. Then, excessive free capture probes displace the enhancement probe in the hybrids formed between the variant reference nucleic acids and the enhancement probes because the hybrids formed between the capture probes and the variant target nucleic acids are more stable than the hybrids formed between the enhancement probes and the variant target nucleic acids. In the same way, excessive free enhancement probes displace the capture probe in the hybrids formed between the reference target nucleic acids and the capture probes because the hybrids formed between the enhancement probes and the reference target nucleic acids are more stable than the hybrids formed between the capture probes and the reference target nucleic acids.

FIG. 5 shows fragment analysis results as follows. FIG. 5(A) shows the fragment analysis result generated from direct amplification of a contrived DNA sample containing 10% mutated A7 DNA without enrichment. FIG. 5(B) shows the fragment analysis result generated when the capture beads were used to enrich mutated A7 alleles. FIG. 5(C) shows the result generated when the capture beads were used to enrich mutated A7 alleles from the contrived DNA sample in the presence of the competing probe. FIG. 5(D) shows the result when the capture beads were used to enrich mutated A7 alleles from the contrived DNA sample in the presence of the enhancement probe.

FIG. 6 shows sequencing results as follows. FIG. 6(A) shows the result when we directly sequenced a contrived DNA sample containing 2% mutated Braf V600E DNA without enrichment. FIG. 6(B) shows the result when the capture beads were used to enrich mutated Braf V600E DNA from the contrived DNA sample containing 2% mutated Braf V600E DNA in the presence of the enhancement probe.

FIG. 7 shows sequencing results as follows. FIG. 7(A) shows the result when we directly sequenced a contrived DNA sample containing G12D mutation without enrichment. FIG. 7(B) shows the result when the capture beads containing all seven capture probes were used to enrich mutated G12D DNA from a contrived DNA sample containing 0.5% mutated G12D DNA in the presence of the enhancement probe. FIG. 7(C) shows the result when we directly sequenced a contrived DNA sample containing G12C mutation without enrichment. FIG. 7(D) shows the result when the capture beads containing all seven capture probes were used to enrich mutated G12C DNA from a contrived DNA sample containing 0.5% mutated G12C DNA in the presence of enhancement probe. FIG. 7(E) shows the result when we directly sequenced a contrived DNA sample containing G13D mutation without enrichment. FIG. 7(F) shows the result when the capture beads containing all seven capture probes were used to enrich mutated G13D from a contrived DNA sample containing 0.5% mutated G13D DNA in the presence of the enhancement probe.

FIG. 8 shows sequencing results generated when four different hybridization buffers were used. Results for FIGS. 8(A), 8(B), 8(C) and 8(D) were obtained with 2M GuSCN, 500 mM NaCl, 10 mM MgCl₂, or 5×PBS buffer, respectively.

FIG. 9 shows sequencing results as follows. FIG. 9(A) shows the result when we directly sequenced a contrived DNA sample containing 2% mutated Braf V600E DNA without enrichment. FIGS. 9(B), 9(C), and 9(D) shows the results when the nucleic acid probe composition, systems, and methods that involved liquid phase hybridization were used to enrich mutated Braf V600E DNA from the contrived DNA sample containing 2% mutated Braf V600E DNA, with hybridization buffers of 2M GuSCN, 500 mM NaCl, and 10 mM MgCl₂, respectively.

FIG. 10 shows sequencing results as follows. FIG. 10(A) shows the result when no enhancement probes are present, where no mutated G12D was detected. FIG. 10(B) shows the result when enhancement probes are present, wherein mutated G12D was detected.

FIG. 11 shows sequencing results as follows. Results for FIG. 11(A) were obtained by mixing the capture probes (beads) and contrived DNA sample, which contained 0.5% of mutated G12D DNA, together for 60 min for hybridization in the absence of enhancement probes. Then, the capture beads were isolated and washed. Finally, extracted DNA was eluted from the beads and subjected to Sanger sequencing. Results for FIG. 11(B) were obtained by mixing the capture probes (beads) and same contrived DNA sample together for 60 min for hybridization, followed by adding enhancement probes to the mixture for incubation for another 30 min. Then, the capture beads were isolated and washed. Finally, extracted DNA was eluted from the beads and subjected sanger sequencing.

FIG. 12 shows sequencing results as follows. FIG. 12(A) shows results for DNA extracted from an FFPE sample without enrichment by toehold displacement/toehold exchange. FIG. 12(B) shows results for DNA extracted from an FFPE sample with enrichment by toehold displacement/toehold exchange.

FIG. 13 shows sequencing results as follows. FIG. 13(A) shows results for DNA extracted from a serum sample without enrichment by toehold displacement/toehold exchange. FIG. 13(B) shows results for DNA extracted from a serum sample with enrichment by toehold displacement/toehold exchange.

FIG. 14 shows sequencing results as follows. FIGS. 14(A), (B), and (C) show results when the capture probe comprises a mismatched nucleotide of T (FIG. 14(A)), C (FIG. 14(B)), and G (FIG. 14(C)), respectively. FIG. 14(D) shows results when both the capture and enhancement probes comprise three (3) mismatched nucleotides. FIG. 14(E) shows results when both the capture and enhancement probes comprise five (5) mismatched nucleotides.

### DETAILED DESCRIPTION OF THE INVENTION

A nucleic acid probe composition for enriching, extracting, and detecting a variant target nucleic acid in a sample comprising the variant target nucleic acid and a reference target nucleic acid is disclosed. Without wishing to be bound by theory, we believe that a substantial improvement in analytical sensitivity of detection of a variant target nucleic acid in a sample comprising the variant target nucleic acid and a reference target nucleic acid can be achieved by use of the nucleic acid probe composition, systems, and methods disclosed herein. This can be accomplished by designing capture probes and enhancement probes for hybridization to variant and reference target nucleic acids, respectively, in which the capture probe includes a 5′ upstream or 3′ downstream toehold domain, and conversely the enhancement probe includes a 3′ downstream or 5′ upstream toehold domain, and in which the capture probe binds the intermediate subsequence of a strand segment of the variant target nucleic acid more strongly than the intermediate subsequence of a strand segment of the reference target nucleic acid, and conversely the enhancement probe binds the intermediate subsequence of a strand segment of the reference target nucleic acid more strongly than the intermediate subsequence of a strand segment of the variant target nucleic acid, and then using the capture and enhancement probes for enriching the variant target nucleic acid in the sample before carrying out detection of the variant target nucleic acid. The nucleic acid probe composition, systems, and methods also can be used to specifically capture variant target nucleic acids or reference target nucleic acids without capturing both.

Importantly, enrichment of the variant target nucleic acid relative to the reference target nucleic acid of as little as about 10 fold can be sufficient to make mutation analysis practical in biologically relevant samples, such as blood samples, in which the variant target nucleic acid is present at concentrations too low to be detectable accurately or precisely by conventional amplification process.

This is because replication errors associated with amplification are a primary factor limiting analytical sensitivity of tests to detect variant target nucleic acids that are present in samples at low concentrations relative to reference target nucleic acids. Replication errors alter the DNA sequence of amplification products, thereby obscuring the identities of the targets that are present at low concentrations.

To illustrate, in tests carried out according to the method disclosed herein, to the extent that mutated DNA and wild-type DNA are both present in an initial source, and the mutated DNA is present at a substantially lower concentration than the wild-type DNA, both the mutated and wild-type DNA can be extracted together from the initial source to yield a purified DNA sample containing both the mutated and wild-type DNA. The purified DNA sample may then be subjected to amplification to determine if the mutated DNA is present.

Unfortunately, though, when the replication error associated with amplification is larger than the ratio of the concentration of the mutated DNA to the concentration of the wild-type DNA, analytical sensitivity is limited.

Considering this, we realized that the replication error effect on analytical sensitivity can be minimized if the concentration of mutated DNA concentration is increased—before amplification—without altering the original DNA sequence, to a level at which ratio of the concentration of the mutated DNA to the wild-type DNA is larger than the replication error. This can be achieved by using the nucleic acid probe composition, systems, and methods disclosed herein because the composition, systems, and methods can be used to enrich the mutated DNA of the sample without altering the original DNA sequence.

To further illustrate the impact of the nucleic acid probe composition, systems, and methods, let us say we want to detect 0.2% mutated DNA (i.e., the ratio of the concentration of the mutated DNA to the concentration of the wild-type DNA is 0.002), but the replication error of the test is 1%, then the replication error of the test is too high for the test to detect the mutated DNA accurately and precisely. However, if the nucleic acid probe composition, systems, and methods are used to enrich the mutated DNA by as little as about 10 fold (meaning that the mutated DNA concentration increases from 0.2% to 2%) before amplification, the test then can detect the mutated DNA accurately and precisely, because the mutated DNA concentration is now 2%, larger than the replication error of 1%.

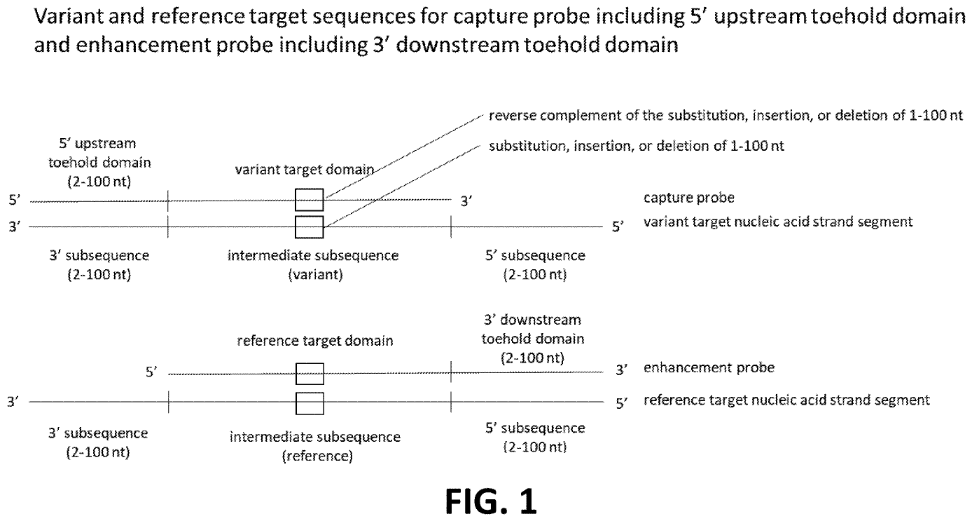

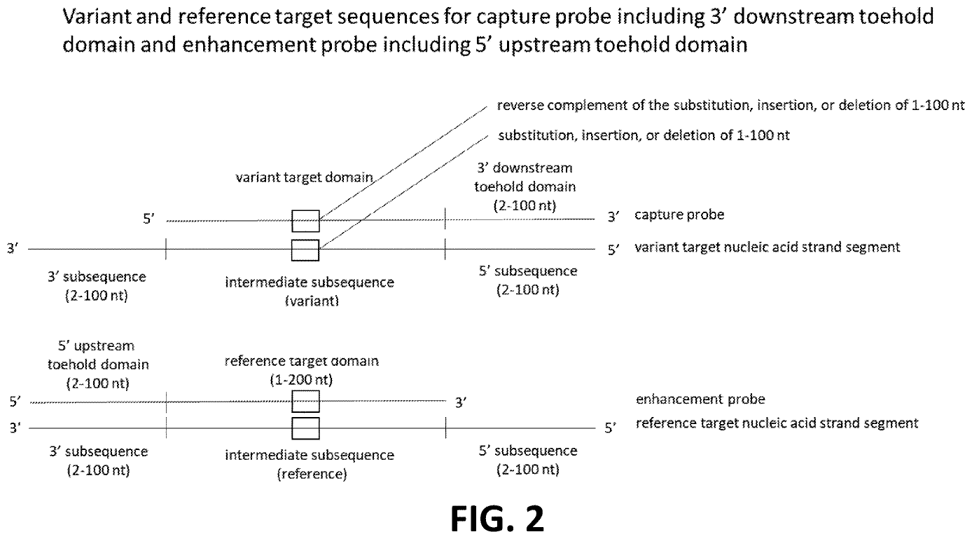

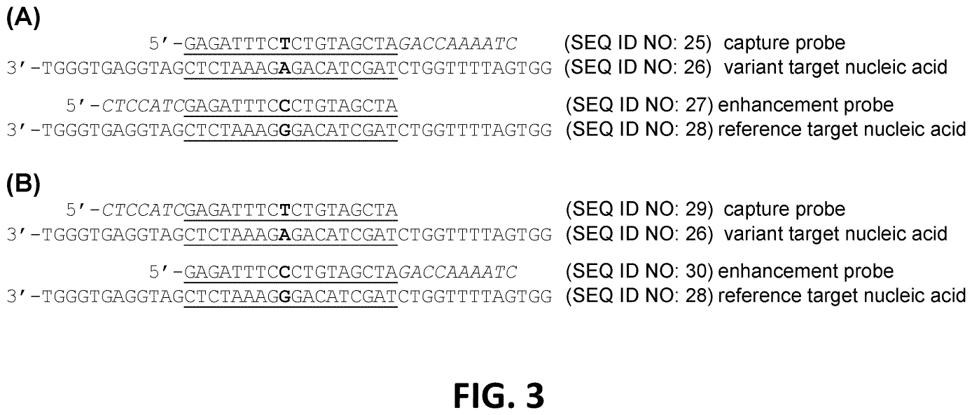

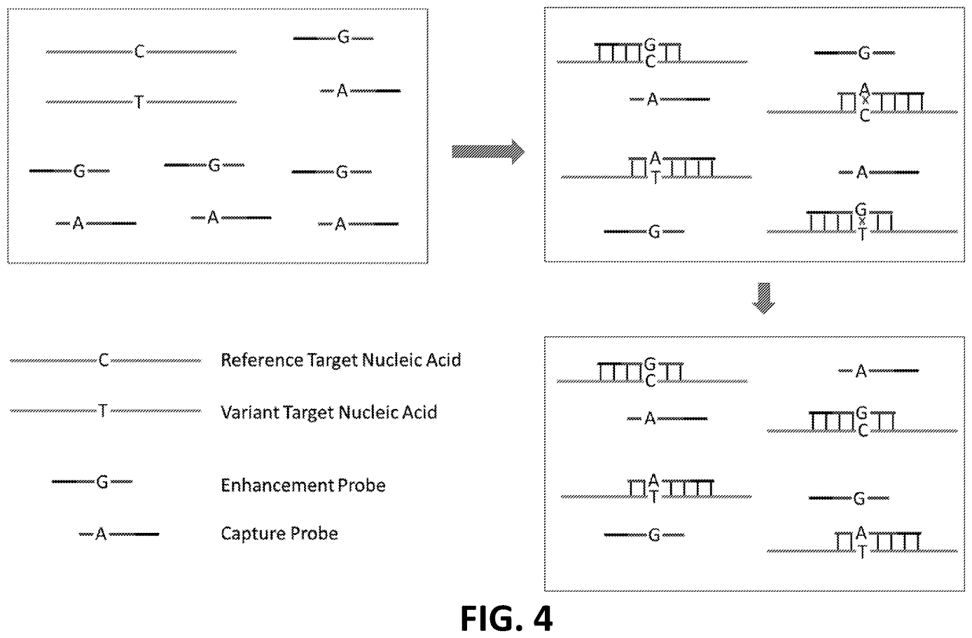

Considering the variant and reference target nucleic acids in detail, with reference to FIGS. 1-4, each of the variant and reference target nucleic acids comprise a nucleic acid strand segment having a 5′ end, a 3′ end, and a nucleic acid sequence. The strand segments of the variant and reference target nucleic acids can be, for example, segments of sequences within larger nucleic acid molecules in a sample. For example, the strand segments of the variant and reference target nucleic acids can be subsequences within a DNA sequence of a gene, a plasmid, a viral genome, and/or a chromosome. Thus, the nucleic acid probe composition, systems, and methods disclosed herein can be used to detect a sequence variant within a discrete segment of a much larger nucleic acid. Alternatively or additionally, the strand segments of the variant and reference target nucleic acids can be, for example, segments of sequences separate and distinct from other nucleic acid molecules in a sample. For example, the strand segments of the variant and reference target nucleic acids can be DNA fragments separate and distinct from other nucleic acid molecules in a sample. Thus, the nucleic acid probe composition, systems, and methods disclosed herein also can be used to detect a sequence variant occurring in a short segment of a nucleic acid, such as a sequence of 100-200 nucleotides, which can be either single-stranded or double-stranded.

Unless specified, the target nucleic acid means one of the variant and reference target nucleic acids or both.

In some examples, the nucleic acid sequences of the strand segments of the variant and reference target nucleic acids each comprise 15 to 400 nucleotides, 18 to 250 nucleotides, 19 to 160 nucleotides, 21 to 110 nucleotides, 22 to 70 nucleotides, or 24 to 45 nucleotides. Variant target nucleic acids in strand segments with lengths specified in the narrower ranges, e.g., 24 to 45 nucleotides, may be easier to detect than those in strand segments with lengths specified in the broader ranges, e.g., 15 to 400 nucleotides.

The nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid.

In some examples, the substitution, insertion, or deletion comprises one or more of a single-base substitution, a multiple-base substitution, a single-base insertion, a multiple-base insertion, a single-base deletion, or a multiple-base deletion.

In some examples the substitution, insertion, or deletion comprises 1 to 90 nucleotides, 1 to 80 nucleotides, 1 to 70 nucleotides, 1 to 60 nucleotides, 1 to 50 nucleotides, 1 to 40 nucleotides, 1 to 30 nucleotides, 1 to 20 nucleotides, 1 to 10 nucleotides, 1 to 9 nucleotides, 1 to 8 nucleotides, 1 to 7 nucleotides, 1 to 6 nucleotides, 1 to 5 nucleotides, 1 to 4 nucleotides, 1 to 3 nucleotides, or 1 or 2 nucleotides.

Considering the composition in detail, the composition comprises a capture probe comprising a single-stranded oligonucleotide that has a 5′ end and a 3′ end and comprises a variant target domain and either a 5′ upstream toehold domain or a 3′ downstream toehold domain, but not both, contiguous to the variant target domain. The oligonucleotide of the capture probe is a single-stranded oligonucleotide, and thus the capture probe can be a single-stranded oligonucleotide, as distinct from a double-stranded oligonucleotide. Regarding the variant target domain being contiguous to the 5′ upstream toehold domain or the 3′ downstream toehold domain, this means that for a capture probe comprising a single-stranded oligonucleotide that comprises a 5′ upstream toehold domain, the 5′ upstream toehold domain is immediately upstream of the variant target domain, such that the variant target domain is attached to the 5′ upstream toehold domain directly, without any other sequence intervening. This also means that for a capture probe comprising a single-stranded oligonucleotide that comprises a 3′ downstream toehold domain, the 3′ upstream toehold domain is immediately downstream of the variant target domain, such that the variant target domain is attached to the 3′ downstream toehold domain directly, without any other sequence intervening.

The composition also comprises an enhancement probe comprising a single-stranded oligonucleotide that has a 5′ end and a 3′ end and comprises a reference target domain and either a 3′ downstream toehold domain or a 5′ upstream toehold domain, but not both, contiguous to the reference target domain. Like for the capture probe, the oligonucleotide of the enhancement probe is a single-stranded oligonucleotide, and thus the enhancement probe also can be a single-stranded oligonucleotide, as distinct from a double-stranded oligonucleotide. Also like for the capture probe, for an enhancement probe comprising a single-stranded oligonucleotide that comprises a 5′ upstream toehold domain, the 5′ upstream toehold domain is immediately upstream of the reference target domain, such that the reference target domain is attached to the 5′ upstream toehold domain directly, without any other sequence intervening. Likewise, for an enhancement probe comprising a single-stranded oligonucleotide that comprises a 3′ downstream toehold domain, the 3′ downstream toehold domain is immediately downstream of the reference target domain, such that the reference target domain is attached to the 3′ downstream toehold domain directly, without any other sequence intervening.

In some examples, the variant target domain and the reference target domain each comprise 1 to 200 nucleotides, 2 to 150 nucleotides, 3 to 80 nucleotides, 5 to 50 nucleotides, 10 to 30 nucleotides or 16 to 25 nucleotides. Variant target domains and reference target domains with sizes specified in the narrower ranges, e.g., 16 to 25 nucleotides, may be more useful for detection than those with sizes specified in the broader ranges, e.g., 1 to 200 nucleotides.

In some embodiments, the capture probe and the enhancement probe each comprise 7 to 300 nucleotides, 13 to 200 nucleotides, 17 to 120 nucleotides, 18 to 80 nucleotides, 19 to 50 nucleotides, or 20 to 35 nucleotides. Capture probes and enhancement probes with sizes specified in the narrower ranges, e.g., 20 to 35 nucleotides, may be more useful for detection than those with sizes specified in the broader ranges, e.g., 7 to 300 nucleotides.

The single-stranded oligonucleotides of the capture probe and the enhancement probes can comprise, for example, DNA, RNA, locked nucleic acids, or peptide nucleic acids, or a combination thereof.

Thus, in some examples the single-stranded oligonucleotide of the capture probe comprises one or more of DNA, RNA, locked nucleic acids, or peptide nucleic acids, or a combination thereof, and the single-stranded oligonucleotide of the enhancement probe comprises one or more of DNA, RNA, locked nucleic acids, or peptide nucleic acids, or a combination thereof.

The 5′ upstream toehold domain has a nucleic acid sequence that is a reverse complement of 3′ subsequences of the nucleic acid sequences of the strand segments of the variant and reference target nucleic acids extending from the 3′ ends of the strand segments of the variant and reference target nucleic acids. Also, the 3′ downstream toehold domain has a nucleic acid sequence that is a reverse complement of 5′ subsequences of the nucleic acid sequences of the strand segments of the variant and reference target nucleic acids extending from the 5′ ends of the strand segments of the variant and reference target nucleic acids.

The variant target domain has a nucleic acid sequence that is a reverse complement of an intermediate subsequence of the strand segment of the variant target nucleic acid that extends contiguously between the 5′ and 3′ subsequences of the strand segment of the variant target nucleic acid and comprises the substitution, insertion, or deletion, or combination thereof.

The reference target domain has a nucleic acid sequence that is a reverse complement of an intermediate subsequence of the strand segment of the reference target nucleic acid that extends contiguously between the 5′ and 3′ subsequences of the strand segment of the reference target nucleic acid and does not comprise the substitution, insertion, or deletion, or combination thereof.

In some examples, the reverse complement can be a nucleic acid sequence that comprises one or more mismatched nucleotides in the positions other than those where the substitution, insertion or deletion occurs, and is not fully complementary to the corresponding sequence of the variant or reference target nucleic acid sequence.

In other examples, the reverse complement can be a nucleic acid sequence that is fully complementary to the corresponding sequence of the variant or reference target nucleic acid sequence.

For example, in some examples, a capture probe containing mismatched nucleotides comprises at least 6 pairs of adjacent matched nucleotides with respect to the nucleic acid sequence of the strand segment of the corresponding variant target nucleic acid. The following example explains how to count adjacent matched nucleotide pairs in a sequence. Let us say that C, T, and A are matched nucleotides and G is a mismatched nucleotide in a sequence of CTGA. This sequence has only one pair of adjacent matched nucleotides (CT). In contrast, if C, T, and G are matched nucleotides and A is a mismatched nucleotide in the sequence of CTGA, then there are two pairs of adjacent matched nucleotides (CT and TG). The number of adjacent matched nucleotides sufficient for a capture probe depends on the form of the capture probe. For capture probes comprising locked nucleic acids, as few as 6 adjacent matched nucleotides can be sufficient. For capture probes comprising GC rich DNA, e.g., DNA with 100% GC-content, as few as 8 adjacent matched nucleotides can be sufficient. For capture probes comprising AT rich DNA, e.g., DNA with less than 50% GC content, 16 adjacent matched nucleotides can be sufficient.

Also in some examples, an enhancement probe containing mismatched nucleotides comprises at least 6 pairs of adjacent matched nucleotides with respect to the nucleic acid sequence of the strand segment of the corresponding reference target nucleic acid.

Also in some examples, the capture probe contains only matched nucleotides with respect to the nucleic acid sequence of the strand segment of the corresponding variant target nucleic acid.

Also in some examples, the enhancement probe contains only matched nucleotides with respect to the nucleic acid sequence of the strand segment of the corresponding reference target nucleic acid.

Thus, in some examples, the capture probe comprises at least 6 pairs of adjacent matched nucleotides with respect to the nucleic acid sequence of the strand segment of the variant target nucleic acid, and the enhancement probe comprises at least 6 pairs of adjacent matched nucleotides with respect to the nucleic acid sequence of the strand segment of the reference target nucleic acid.

Also in some examples, the capture probe contains only matched nucleotides with respect to the nucleic acid sequence of the strand segment of the variant target nucleic acid, and the enhancement probe contains only matched nucleotides with respect to the nucleic acid sequence of the strand segment of the reference target nucleic acid.

Like the variant target domain and the reference target domain, in some examples the intermediate subsequence of the strand segment of the variant target nucleic acid and the intermediate subsequence of the strand segment of the reference target nucleic acid each comprise 1 to 200 nucleotides, 2 to 150 nucleotides, 3 to 80 nucleotides, 5 to 50 nucleotides, 10 to 30 nucleotides or 16 to 25 nucleotides.

If the capture probe comprises the 5′ upstream toehold domain, then the enhancement probe comprises the 3′ downstream toehold domain, whereas if the capture probe comprises the 3′ downstream toehold domain, then the enhancement probe comprises the 5′ upstream toehold domain.

The nucleic acid sequences of the 5′ upstream toehold domain and the 3′ downstream toehold domain each comprise 2 to 100 nucleotides. As discussed above, toehold displacement involves an incoming nucleic acid strand outcompeting and thereby displacing an incumbent strand of a nucleic acid duplex to form a better matched duplex with a complementary remaining strand of the duplex, and this is accomplished based on the incoming strand having a unique toehold domain that includes about 4 to 10 nucleotides, is complementary to the remaining strand, and is not present in the incumbent strand. Also as discussed above, toehold displacement is controlled by the Gibbs free energy of hybridization, the initial step of the incoming strand binding the remaining strand by the toehold is endothermic and rate limiting, and the step can be tuned over a range of six orders of magnitude by varying the strength of the toehold domain, for example based on varying its length and/or G/C content. Analogously here, hybridization of the 5′ upstream toehold domain and the 3′ downstream toehold domain, each including 2 to 100 nucleotides, to their respective 3′ and 5′ subsequences of the strand segments of the variant and reference target nucleic acids can be tuned by varying the strength of the toehold domains, for example based on varying their lengths and/or G/C content. For example, short toehold domains, e.g., including only 2, 3, or 4 nucleotides, can be made using G and/or C bases for stronger toehold domains than would be obtained with the same number of A and/or T bases.

In some examples, the 5′ upstream toehold domain and the 3′ downstream toehold domain each comprise 2 to 90 nucleotides, 2 to 80 nucleotides, 2 to 70 nucleotides, 2 to 60 nucleotides, 2 to 50 nucleotides, 2 to 40 nucleotides, 3 to 30 nucleotides, 3 to 20 nucleotides, 4 to 16 nucleotides, 4 to 14 nucleotides, or 4 to 10 nucleotides. 5′ upstream toehold domains and the 3′ downstream toehold domains with sizes specified in the narrower ranges, e.g., 4 to 10 nucleotides, may be more useful for detection than those with sizes specified in the broader ranges, e.g., 2 to 90 nucleotides.

The capture probe and the enhancement probe must be designed so that the capture probe, specifically the variant target domain of the capture probe, binds the intermediate subsequence of the strand segment of the variant target nucleic acid more strongly than the intermediate subsequence of the strand segment of the reference target nucleic acid, and conversely so that the enhancement probe, specifically the reference target domain of the enhancement probe, binds the intermediate subsequence of the strand segment of the reference target nucleic acid more strongly than the intermediate subsequence of the strand segment of the variant target nucleic acid. This can be accomplished by analyzing the specific sequences of the relevant variant and reference target nucleic acid sequences and designing the capture probe and the enhancement probe such that they can selectively bind to the intermediate subsequences of the strand segments of the variant and reference target nucleic acids, respectively.

In some examples, the composition can be used for detection of a single variant target nucleic acid in a sample comprising the variant target nucleic acid and a single reference target nucleic acid. In accordance with these examples, the composition can comprise a single capture probe and a single enhancement probe. In these examples, the single capture probe comprises a variant target domain that has a nucleic acid sequence that is a reverse complement of the intermediate subsequence of the strand segment of the single variant target nucleic acid that comprises the substitution, insertion, or deletion, or combination thereof, of the single variant target nucleic acid. Also in these examples, the single enhancement probe comprises a reference target domain that has a nucleic acid sequence that is a reverse complement of an intermediate subsequence of the strand segment of the reference target nucleic acid that does not comprise the substitution, insertion, or deletion, or a combination thereof. Thus, in some examples the capture probe comprises only one capture probe, and the enhancement probe comprises only one enhancement probe, wherein the capture probe is specific to a single variant target nucleic acid, and the enhancement probe is specific to a single reference target nucleic acid.

In some examples, the composition can be used for detection of a plurality of variant target nucleic acids in a sample comprising the plurality of variant target nucleic acids and a single reference target nucleic acid. In accordance with these examples, the composition can comprise a plurality of capture probes, for example 2, 3, 4, or more capture probes, each specific to a different variant target nucleic acid, and a single enhancement probe specific to a single reference target nucleic acid, wherein the single reference target nucleic acid is a reference nucleic acid to each of the different variant target nucleic acids. Thus, in some examples the capture probe comprises a plurality of capture probes, and the enhancement probe comprises only one enhancement probe, wherein each of the capture probes is specific to a different variant target nucleic acid, and the enhancement probe is specific to a single reference target nucleic acid.

In some examples, the composition can be used for detection of a plurality of variant target nucleic acids in a sample comprising the plurality of variant target nucleic acids and a plurality of reference target nucleic acids. In accordance with these examples, the composition can comprise a plurality of capture probes, for example 2, 3, 4, or more capture probes, each specific to a different variant target nucleic acid, and a plurality of enhancement probes, for example 2, 3, 4, or more enhancement probes, each specific to a different reference target nucleic, wherein each of the reference target nucleic acids is a reference nucleic acid to one or more of the variant target nucleic acids. Thus, in some examples the capture probe comprises a plurality of capture probes, and the enhancement probe comprises a plurality of enhancement probes, wherein each of the capture probes is specific to a different variant target nucleic acid, and each of the enhancement probes is specific to a different reference target nucleic acid.

The composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe. This means that the composition can be used for enriching, extracting, and detecting one or more variant target nucleic acids in a sample comprising the one or more variant target nucleic acids and one or more reference target nucleic acids without needing any oligonucleotides that are complementary to the 5′ subsequences, the intermediate subsequences, or the 3′ subsequences of the strand segments of the one or more variant or reference target nucleic acids other than the one or more capture probes and the one or more enhancement probes. This also means that the composition can be used for enriching, extracting, and detecting one or more variant target nucleic acids in a sample comprising the one or more variant target nucleic acids and one or more reference target nucleic acids without needing any nucleic acids that are complementary to the one or more capture probes or the one or more enhancement probes other than the strand segments of the one or more variant and reference target nucleic acids. For example, in some examples the composition does not comprise any other oligonucleotides that are complementary to the 5′ subsequences, the intermediate subsequences, or the 3′ subsequences of the strand segments of the variant or reference target nucleic acids and does not comprise any other nucleic acids that are complementary to the capture probe or the enhancement probe. This is advantageous for simplicity in making and using the composition for enriching, extracting, and detecting a variant target nucleic acid in a sample comprising the variant target nucleic acid and a reference target nucleic acid, particularly relative to previous approaches for use of toehold domains.

In some examples, the capture probe and/or the enhancement probe comprise a short sequence of nucleotides at their respective 3′ ends that form a sequence that is not a reverse complement of their respective variant and reference target nucleic acids. The addition of such a short sequence of nucleotides at the 3′ ends of the capture probe and/or the enhancement probe can be advantageous for preventing the capture probe and/or the enhancement probe from being elongated by PCR, to the extent that PCR may be used in the methods, improving accuracy and precision of the methods. In some examples the capture probe and the enhancement probe include the same short sequence of nucleotides at their respective 3′ ends. In some examples the capture probe and the enhancement probe include different short sequences of nucleotides at their respective 3′ ends. In some examples the short sequence of nucleotides includes 3, 4, 5, 6, 7, 8, 9, or 10 nucleotides. In some examples the short sequence of nucleotides is TTTT or AAAA.

Also in some examples, the capture probe and/or the enhancement probe comprise one or more nucleotides at their respective 3′ end that are modified for preventing the capture probe and/or the enhancement probe from being elongated by PCR.

In some examples the composition further comprises a guanidine-based salt. Surprisingly, we have observed that including a guanidine-based salt in the composition substantially improves enrichment, extraction, and detection of variant target nucleic acids in comparison to conventional buffers for hybridization of nucleic acids that include sodium chloride (NaCl), magnesium chloride (MgCl₂), and/or phosphate buffered saline (PBS), but that lack a guanidine-based salt. Without wishing to be bound by theory, it is believed that the presence of a guanidine-based salt, such as guanidinium isothiocyanate (GuSCN), improves enrichment and thus improves detection.

In some of these examples, the guanidine-based salt comprises one or more of guanidine or a guanidine derivative with the structure (R1R2N)(R3R4N)C═N—R5, wherein R1, R2, R3, R4, and R5 each independently are either (i) hydrogen or (ii) a functional group comprising 1 to 4 carbons with hydrogens, with the proviso that the sum of carbons in R1 to R5 is less than or equal to 15.

Also in some of these examples, the composition is aqueous and the guanidine-based salt is present in the composition at a concentration of 0.05 to 8 M. The guanidine-based salt can be present in the composition, for example at a concentration of 0.5 to 4 M, 1.0 to 3.0 M, or 1.5 to 2.5 M, 1.6 to 2.4 M, 1.7 to 2.3 M, 1.8 to 2.2 M, 1.9 to 2.1 M, or about 2.0 M.

Also in some of these examples, the guanidine-based salt comprises guanidinium isothiocyanate, e.g., at a concentration of 0.05 to 8 M, 0.5 to 4 M, 1.0 to 3.0 M, or 1.5 to 2.5 M, 1.6 to 2.4 M, 1.7 to 2.3 M, 1.8 to 2.2 M, 1.9 to 2.1 M, or about 2.0 M.

In some examples, the composition does not comprise the variant or reference target nucleic acids. This is advantageous when preparing the composition for storage and future use. In some examples, the composition comprises one or more of the variant and reference target nucleic acids. This is advantageous for using the composition for enriching, extracting, and detecting the variant target nucleic acid in a sample comprising the variant target nucleic acid and the reference target nucleic acid.

A system for enriching, extracting, and detecting a variant target nucleic acid in a sample comprising the variant target nucleic acid and a reference target nucleic acid also is disclosed. The variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid.

The system comprises:
(a) the composition for enriching, extracting, and detecting a variant target nucleic acid in a sample as described above; and
(b) a solid.

The capture probe is immobilized on a surface of the solid.

Another system for enriching, extracting, and detecting a variant target nucleic acid in a sample comprising the variant target nucleic acid and a reference target nucleic acid also is disclosed. The variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid.

The system comprises:
(a) the composition for enriching, extracting, and detecting a variant target nucleic acid in a sample as described above; and
(b) a solid.

The capture probe comprises at least one binding tag.

The solid comprises an extraction tag that binds to the at least one binding tag.

A method for enriching, extracting, and detecting a variant target nucleic acid in a sample comprising the variant target nucleic acid and a reference target nucleic acid also is disclosed. The variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid.

The method comprises steps of:
(1) combining the sample, a capture probe, and an enhancement probe, thereby forming a hybridization composition;
(2) incubating the hybridization composition at a temperature that promotes the formation of hybrids of the capture probe and the variant target nucleic acid and hybrids of the enhancement probe and the reference target nucleic acid by toehold displacement and/or toehold exchange;
(3) isolating the hybrids formed by the capture probe from the hybrids formed by the enhancement probe;
(4) eluting the variant target nucleic acid from the hybrids of the capture probe and the variant target nucleic acid; and
(5) detecting the variant target nucleic acid.

The capture probe comprises a single-stranded oligonucleotide that has a 5′ end and a 3′ end and comprises a variant target domain and either a 5′ upstream toehold domain or a 3′ downstream toehold domain, but not both, contiguous to the variant target domain.

The enhancement probe comprises a single-stranded oligonucleotide that has a 5′ end and a 3′ end and comprises a reference target domain and either a 3′ downstream toehold domain or a 5′ upstream toehold domain, but not both, contiguous to the reference target domain.

The 5′ upstream toehold domain has a nucleic acid sequence that is a reverse complement of 3′ subsequences of the nucleic acid sequences of the strand segments of the variant and reference target nucleic acids extending from the 3′ ends of the strand segments of the variant and reference target nucleic acids.

The 3′ downstream toehold domain has a nucleic acid sequence that is a reverse complement of 5′ subsequences of the nucleic acid sequences of the strand segments of the variant and reference target nucleic acids extending from the 5′ ends of the strand segments of the variant and reference target nucleic acids.

The variant target domain has a nucleic acid sequence that is a reverse complement of an intermediate subsequence of the strand segment of the variant target nucleic acid that extends contiguously between the 5′ and 3′ subsequences of the strand segment of the variant target nucleic acid and comprises the substitution, insertion, or deletion, or combination thereof.

The reference target domain has a nucleic acid sequence that is a reverse complement of an intermediate subsequence of the strand segment of the reference target nucleic acid that extends contiguously between the 5′ and 3′ subsequences of the strand segment of the reference target nucleic acid and does not comprise the substitution, insertion, or deletion, or combination thereof.

If the capture probe comprises the 5′ upstream toehold domain, then the enhancement probe comprises the 3′ downstream toehold domain, whereas if the capture probe comprises the 3′ downstream toehold domain, then the enhancement probe comprises the 5′ upstream toehold domain.

The nucleic acid sequences of the 5′ upstream toehold domain and the 3′ downstream toehold domain each comprise 2 to 100 nucleotides; and

The hybridization composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

In some examples, the method further comprises a step (0) of combining the sample and the capture probe, thereby forming an initial sample-capture probe composition prior to step (1).

In some examples, the method further comprises a step (0) of combining the sample and the enhancement probe, thereby forming an initial sample-enhancement probe composition prior to step (1).

In some examples, (i) the method further comprises a step (0) of immobilizing the capture probe on a surface of a solid prior to step (1); and (ii) step (3) comprises isolating the hybrids formed by the capture probe from the hybrids formed by the enhancement probe by separating the solid from the hybridization composition.

As noted, the method comprises the step (2) of incubating the hybridization composition at a temperature that promotes the formation of hybrids of the capture probe and the variant target nucleic acid and hybrids of the enhancement probe and the reference target nucleic acid by toehold displacement and/or toehold exchange. Due to complementarity of the capture probe and the enhancement probe to both the variant target nucleic acid and the reference target nucleic acid, the capture probe and the enhancement probe each can form hybrids with either the variant target nucleic acid or the reference target nucleic acid. The step (2) relies on toehold displacement and/or toehold exchange to promote the formation of hybrids of the capture probe and the variant target nucleic acid and also to promote the formation of hybrids of the enhancement probe and the reference target nucleic acid.

Also as noted, the method comprises the step (3) of isolating the hybrids formed by the capture probe from the hybrids formed by the enhancement probe. The isolation accomplishes an increase in the ratio of the concentration of the hybrids formed by the capture probe to the concentration of the hybrids formed by the enhancement probe.

In some examples, (i) the capture probe comprises at least one binding tag; and (ii) step (3) comprises isolating the hybrids formed by the capture probe from the hybrids formed by the enhancement probe by contacting the hybridization solution with a solid that comprises an extraction tag that binds to the at least one binding tag, then separating the solid from the hybridization composition.

Also as noted, the method comprises the step (4) of eluting the variant target nucleic acid from the hybrids of the capture probe and the variant target nucleic acid. The elution can be accomplished by various elution methods known in the art. The elution provides the variant target nucleic acid in a state amenable to subsequent detection.

Also as noted, the method comprises a step (5) of detecting the variant target nucleic acid. The detection can be accomplished by various DNA analysis methods known in the art. Thus, in some examples, step (5) comprises detecting the variant target nucleic acid by a DNA analysis method comprising one or more of real-time PCR, microarray, fragment size analysis, Sanger sequencing, next-generation sequencing, or digital PCR, or a combination thereof.

The method can be applied to the nucleic acid probe composition and systems as described above.

Thus, in some examples the oligonucleotide of the capture probe comprises one or more of DNA, RNA, locked nucleic acids, or peptide nucleic acids, or a combination thereof; and the oligonucleotide of the enhancement probe comprises one or more of DNA, RNA, locked nucleic acids, or peptide nucleic acids, or a combination thereof.

In some examples, the substitution, insertion, or deletion comprises one or more of a single-base substitution, a multiple-base substitution, a single-base insertion, a multiple-base insertion, a single-base deletion, or a multiple-base deletion.

In some examples, the hybridization solution further comprises a guanidine-based salt. In some of these examples, the guanidine-based salt comprises one or more of guanidine or a guanidine derivative with the structure (R1R2N)(R3R4N)C═N—R5, wherein R1, R2, R3, R4, and R5 each independently are either (i) hydrogen or (ii) a functional group comprising 1 to 4 carbons with hydrogens, with the proviso that the sum of carbons in R1 to R5 is less than or equal to 15. Also in some of these examples, the guanidine-based salt is present in the hybridization composition at a concentration of 0.05 to 8 M. Also in some of these examples, the guanidine-based salt comprises guanidinium isothiocyanate, e.g., at a concentration of 0.05 to 8 M, 0.5 to 4 M, 1.0 to 3.0 M, or 1.5 to 2.5 M, 1.6 to 2.4 M, 1.7 to 2.3 M, 1.8 to 2.2 M, 1.9 to 2.1 M, or about 2.0 M.

In some examples, the nucleic acid sequences of the strand segments of the variant and reference target nucleic acids each comprise 15 to 400 nucleotides.

In some examples, the variant target domain and the reference target domain each comprise 1 to 200 nucleotides.

In some examples, the capture probe comprises only one capture probe and the enhancement probe comprises only one enhancement probe, wherein the capture probe is specific to a single variant target nucleic acid, and the enhancement probe is specific to a single reference target nucleic acid. In some examples, the capture probe comprises a plurality of capture probes, and the enhancement probe comprises only one enhancement probe, wherein each of the capture probes is specific to a different variant target nucleic acid, and the enhancement probe is specific to a single reference target nucleic acid. In some examples, the capture probe comprises a plurality of capture probes, and the enhancement probe comprises a plurality of enhancement probes, wherein each of the capture probes is specific to a different variant target nucleic acid, and each of the enhancement probes is specific to a different reference target nucleic acid, wherein each of the reference target nucleic acids is a reference nucleic acid to one or more of the variant target nucleic acids.

In some examples, the capture probe comprises at least 6 pairs of adjacent matched nucleotides with respect to the nucleic acid sequence of the strand segment of the variant target nucleic acid, and the enhancement probe comprises at least 6 pairs of adjacent matched nucleotides with respect to the nucleic acid sequence of the strand segment of the reference target nucleic acid.

In some examples, the capture probe contains only matched nucleotides with respect to the nucleic acid sequence of the strand segment of the variant target nucleic acid, and the enhancement probe contains only matched nucleotides with respect to the nucleic acid sequence of the strand segment of the reference target nucleic acid.

In some examples, the hybridization composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

### EXAMPLES

Examples are provided to demonstrate the effectiveness of the nucleic acid probe composition, systems, and methods for enriching, extracting, and detecting variant target nucleic acids.

### Example 1: Enriching Mutated DNA Containing a Deletion Mutation

In this example, we implemented the nucleic acid probe composition, systems, and methods to enrich mutated DNA containing a deletion mutations, specifically mutated “A7” alleles in a background of wild-type “A8” alleles. The ACVR2a gene comprises a run of eight A nucleotides, which can be mutated to an A7 allele by deleting one of the A nucleotides from the run of eight A nucleotides. This example also compares the composition, systems, and methods with prior art compositions, systems, and methods.

In this study, we pre-immobilized a capture probe, ([AmC₆]GTTGTTGTGCATAAAAAAAGA) (SEQ ID NO: 1), which is specific to the mutated A7 allele, onto carboxylate magnetic beads to make capture beads to be used to enrich mutated A7 alleles from a contrived DNA sample containing 10% mutated A7 DNA. The AmC₆ group of the capture probe provides a primary amine that can be immobilized to the carboxylate groups of the magnetic beads with carbodiimide to form an amide bond between the primary amine and the carboxylate groups.

The contrived DNA sample was generated by blending 3,000 copies of mutant A7 DNA and 27,000 copies of wild-type A8 DNA. Both mutant A7 and wild-type A8 DNA were gBlock Gene Fragment DNA, which were obtained from Integrated DNA Technologies.

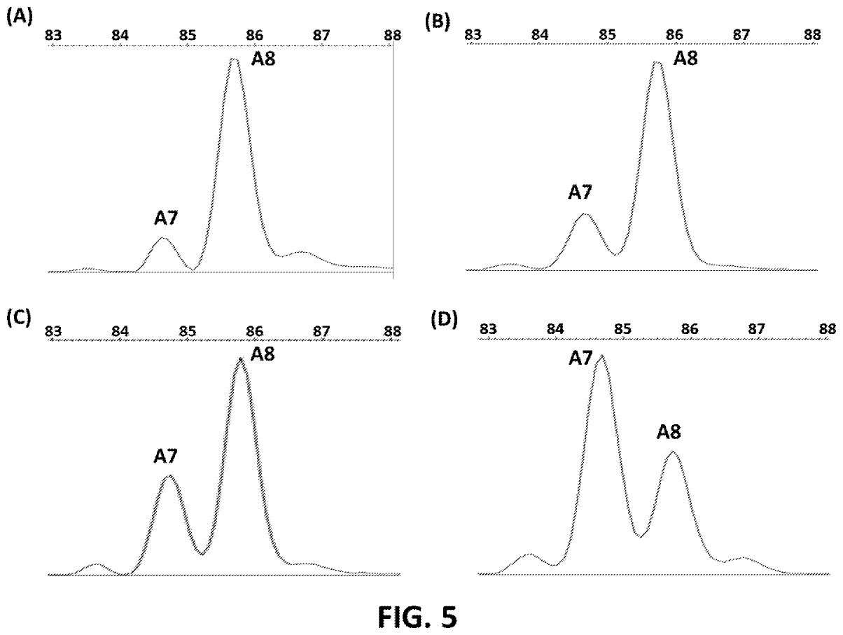

FIG. 5(A) shows the fragment analysis result generated from direct amplification of the contrived DNA sample containing 10% mutated A7 DNA without enrichment, which was intended to be used as a reference to determine the degree of enrichment when the capture beads were utilized to enrich mutated A7 alleles. Experimentally, a contrived DNA sample containing 10% mutated A7 alleles was directly PCR-amplified by LUNA Universal Probe qPCR Master Mix in 20 μL (New England Biolabs). The primers used were FAM-AAAGCTAACTGGATAACTTACAG (SEQ ID NO: 2) (forward) and ACATGCAGGAAGTTGTTGT (SEQ ID NO: 3) (reverse), respectively. The forward primer was labeled by FAM for detection. Finally, the PCR product was subjected to fragment analysis by using a SeqStudio electrophoresis-based sequencer (ThermoFisher). Two major peaks were observed in FIG. 5(A). One arises from mutated A7 alleles, while the other results from wild-type A8 alleles. Of note, the minor peaks appearing in FIG. 5 resulted from PCR spillage.

FIG. 5(B) shows the fragment analysis result generated when the capture beads were used to enrich mutated A7 alleles. Experimentally, we first mixed and incubated the capture beads with the contrived DNA sample for hybridization at room temperature for one hour, in which 2M guanidinium isothiocyanate (GuSCN) was used as hybridization buffer. After hybridization, the magnetic beads were captured and then washed with 2×1M NaCl and 1×50 mM NaCl. Thereafter, captured DNA was eluted from the capture beads, a portion of which was then PCR-amplified by LUNA Universal Probe qPCR Master Mix in 20 μL (New England Biolabs). The primers used were FAM-AAAGCTAACTGGATAACTTACAG (SEQ ID NO: 2) (forward) and ACATGCAGGAAGTTGTTGT (SEQ ID NO: 3) (reverse), respectively. The forward primer was labeled by FAM for detection. Finally, PCR products were subjected to fragment analysis by using a SeqStudio electrophoresis-based sequencer (ThermoFisher). It is seen that the peak intensity ratio of A7 to A8 was slightly larger in FIG. 5(B) than FIG. 5(A), suggesting that a small degree of enrichment was achieved when capture beads containing the capture probes that are specific to mutated A7 alleles were used to enrich mutated A7 alleles.

We next utilized a competing probe that is specific to wild-type A8 alleles to improve enrichment of mutated A7 alleles. This was based on a method of Jeffreys et al. (2003). As noted above, Jeffreys et al. (2003) has reported a method for enriching mutated DNA in which the presence of competing probes that are specific to wild-type alleles could improve enrichment of the mutated DNA. The sequence of the competing probe that we used was GTTGTTGTGCATAAAAAAAAGA (SEQ ID NO: 4). Experimentally, the competing probe was mixed and incubated together with the capture beads and the contrived DNA sample for hybridization at room temperature for one hour. 2M guanidinium isothiocyanate (GuSCN) was used as hybridization buffer. After hybridization, the beads were captured and then washed with 2×1M NaCl and 1×50 mM NaCl. Thereafter, captured DNA was eluted from the capture beads. A portion of eluted DNA was then PCR-amplified by LUNA Universal Probe qPCR Master Mix in 20 μL (New England Biolabs). The primers used were FAM-AAAGCTAACTGGATAACTTACAG (SEQ ID NO: 2) (forward) and ACATGCAGGAAGTTGTTGT (SEQ ID NO: 3) (reverse), respectively. The forward primer was labeled by FAM for detection. Finally, the PCR products were subjected to fragment analysis by using a SeqStudio electrophoresis-based sequencer (ThermoFisher).

FIG. 5(C) shows the result generated when the capture beads were used to enrich mutated A7 alleles from the contrived DNA sample in the presence of the competing probe. It is seen that the peak intensity ratio of A7 to A8 was now larger in FIG. 5(C) than both FIG. 5(A) and FIG. 5(B), suggesting that a better enrichment was indeed achieved in the presence of the competing probe, but the A7 peak intensity is still weaker than the A8 peak intensity.

During the course of our study of enriching mutated A7 alleles, we realized that our nucleic acid probe composition, systems, and methods led to a much better enrichment of mutated A7 alleles than the method of Jeffreys et al. (2003). In our case we used a new probe that we termed “enhancement probe” to replace the competing probe used to produce the results shown in FIG. 5(C). The new enhancement probe has a sequence of CATAAAAAAAAGAGGCCTG (SEQ ID NO: 5), and is specific to wild-type A8 alleles of ACVR2a. Experimentally, the enhancement probe was mixed and incubated together with capture beads and the contrived DNA sample for hybridization at room temperature for one hour, in which 2M GuSCN was used as hybridization buffer. After hybridization, the magnetic beads were retrieved and then washed with 2×1M NaCl and 1×50 mM NaCl. Thereafter, captured DNA was eluted from the capture beads. A portion of eluted DNA was then PCR-amplified by LUNA Universal Probe qPCR Master Mix in 20 μL (New England Biolabs). The primers used were FAM-AAAGCTAACTGGATAACTTACAG (SEQ ID NO: 2) (forward) and ACATGCAGGAAGTTGTTGT (SEQ ID NO: 3) (Reverse), respectively. The forward primer was labeled by FAM for detection. Finally, the PCR products were subjected to fragment analysis by using a SeqStudio electrophoresis-based sequencer (ThermoFisher).

FIG. 5(D) shows the result when the capture beads were used to enrich mutated A7 alleles from the contrived DNA sample in the presence of the enhancement probe. It is seen that in the presence of the enhancement probe, the A7 peak intensity is now even stronger than the A8 peak intensity, suggesting that a much better enrichment of mutated A7 alleles from a contrived DNA sample containing 10% mutated A7 alleles is achieved by the enhancement probe.

### Example 2: Enriching Mutated DNA Containing a Substitution Mutation

This example is provided to demonstrate the effectiveness of the nucleic acid probe composition, systems, and methods in enriching mutated DNA containing a substitution mutation. Specifically, we implemented the composition, systems, and methods to enrich mutated Braf (V600E) DNA in a background of wild-type Braf DNA.

In this study, we first pre-immobilized a capture probe, (GAGATTTCTCTGTAGCTAGACCAAAATCAAAT) (SEQ ID NO: 6), which is specific to mutated Braf V600E DNA, onto magnetic beads to make capture beads to be used to enrich mutated Braf V600E DNA. The short sequence of AAAT at the 3′ end of the capture probe (which is shown in italics in the capture probe sequence) was used to prevent capture probe released from capture beads from being elongated by subsequent PCR. This is because this short sequence is not complementary to the target Braf sequence.

An enhancement probe, CTCCATCGAGATTTCACTGTAGCTATTTT (SEQ ID NO: 7), which is specific to wild-type Braf DNA, was also used. The short sequence of TTTT at the 3′ end of the enhancement probe (which is shown in italics in the capture probe sequence) was used to prevent enhancement probe carried over to PCR from being elongated by PCR as this short sequence is not complementary to the target Braf sequence.

In this example, we enriched mutated Braf V600E alleles from a contrived DNA sample containing 2% mutated Barf V600E DNA. The contrived DNA sample was generated by blending 600 copies of mutant Braf V600E DNA and 29,400 copies of wild-type Braf DNA. Both mutant Braf V600E and wild-type Braf DNA were gBlock Gene Fragment DNA, which were obtained from Integrated DNA Technologies.

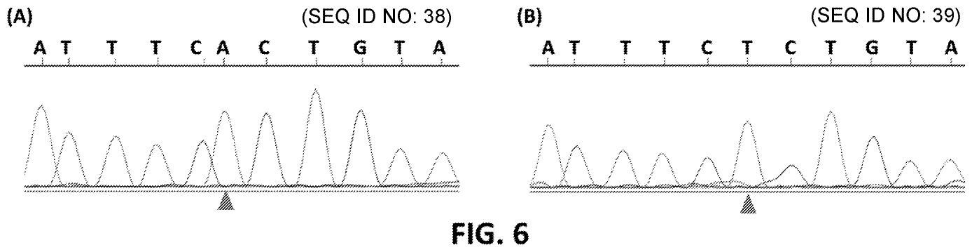

FIG. 6(A) shows the result when we directly sequenced a contrived DNA sample containing 2% mutated Braf V600E DNA without enrichment. This result was used as reference to determine the degree of enrichment when the capture beads were utilized to enrich mutated Braf V600E DNA. Experimentally, a contrived DNA sample containing 2% mutated Braf V600E DNA was directly PCR amplified and sequenced by Sanger sequencing (“Sanger-sequenced”). The primers used for PCR were GACCTCACAGTAAAAATAGGTG (SEQ ID NO: 8) (forward) and ATAGCCTCAATTCTTACCATC (SEQ ID NO: 9) (reverse), respectively. The primer used for Sanger sequencing was ATAGCCTCAATTCTTACCATC (SEQ ID NO: 9).

As seen in FIG. 6(A), the wild-type nucleotide at the V600E mutation site is A pointed by the triangle sign. Clearly, without enrichment, V600E mutation could not be detected from the DNA sample containing 2% mutated Barf V600E DNA by Sanger sequencing.

FIG. 6(B) shows the result when the capture beads were used to enrich mutated Braf V600E DNA from the contrived DNA sample containing 2% mutated Braf V600E DNA in the presence of the enhancement probe. Experimentally, the capture beads, enhancement probe, and contrived DNA sample were mixed and incubated together for hybridization at room temperature for one hour, in which 2M GuSCN was used as hybridization buffer. After hybridization, the magnetic beads were retrieved and then washed with 2×1M NaCl and 1×50 mM NaCl. Thereafter, captured DNA was eluted from capture beads. A portion of eluted DNA was then PCR-amplified and Sanger-sequenced. It is seen that after enrichment by the nucleic acid probe composition, systems, and methods, the mutated nucleotide T pointed by the triangle sign was clearly seen in FIG. 6(B). In fact, the signal of T at the mutation site is at least two times stronger than the signal of the wild-type nucleotide A, suggesting that mutated Braf V600E DNA was significantly enriched from the contrived DNA sample containing 2% mutated Braf V600E DNA, thus becoming detectable by Sanger sequencing. This result shows that the nucleic acid probe composition, systems, and methods also work well when used to enrich and detect mutated DNA containing a substitution mutation.

### Example 3: Scanning the Mutation Status of Multiple Mutations

This example is provided to show the effectiveness of the nucleic acid probe composition, systems, and methods when used to scan the mutation status of multiple potential mutations in a background of wild-type DNA. Specifically, we implemented the nucleic acid probe composition, systems, and methods to scan the mutation status of seven mutations (G12D, G12V, G12A, G12C, G12R, G12S, and G13D) in Codons 12 and 13 of Kras in a background of wild-type DNA. We first pre-immobilized seven capture probes together onto the same magnetic beads to make the capture beads. Each capture probe is specific to one target mutation. TABLE 1 lists the sequence of each capture probe. The short sequence of TTTT at the 3′ end of each capture probe (as shown in italics in each capture probe sequence) was used to prevent the capture probes released from capture beads from being elongated by PCR as this short sequence is not complementary to the target sequence.

In this study, one enhancement probe, which is specific to wild-type Kras DNA, was used to enhance enrichment of each target mutation. The sequence of the enhancement probe also is listed in TABLE 1. The short sequence of TTTT at the 3′ end of the enhancement probe (also shown in italics in the enhancement probe sequence) was used to prevent the enhancement probe carried over to PCR from being elongated as it is not complementary to the target sequence.

We used seven “mutant” gBlock Gene Fragments (one for each mutation) to mimic mutated Kras DNA, and a “wild-type” gBlock Gene Fragment to mimic wild-type DNA. The gBlock Gene Fragment DNA was purchased from Integrated DNA technologies. Each contrived DNA sample contained only one “mutant” gBlock gene Fragment that has one target mutation, in which the mutated DNA abundance was 0.5%. Each contrived DNA sample was generated by blending 600 copies of a specific “mutant” gBlock Gene Fragment and 116,400 copies of “wild-type” gBlock Gene Fragment.

TABLE 1
Sequences of various
probes and primers for Kras

| | | SEQ ID |
|---|---|---|
| Probe or Primer | Sequence | Number |
| Capture Probe | TTGTGGTAGTTGGAGCT | SEQ ID |
| for G12D | GATGGCGTAGGCTTTT | NO: 10 |
| Capture Probe | TTGTGGTAGTTGGAGCT | SEQ ID |
| for G12V | GTTGGCGTAGGCTTTT | NO: 11 |
| Capture Probe | TTGTGGTAGTTGGAGCT | SEQ ID |
| for G12A | GCTGGCGTAGGCTTTT | NO: 12 |
| Capture Probe | TTGTGGTAGTTGGAGCT | SEQ ID |
| for G12C | TGTGGCGTAGGCTTTT | NO: 13 |
| Capture Probe | TTGTGGTAGTTGGAGCT | SEQ ID |
| for G12S | AGTGGCGTAGGCTTTT | NO: 14 |
| Capture Probe | TTGTGGTAGTTGGAGCT | SEQ ID |
| for G12R | CGTGGCGTAGGCTTTT | NO: 15 |
| Capture Probe | TTGTGGTAGTTGGAGCT | SEQ ID |
| for G13D | GGTGACGTAGGCTTTT | NO: 16 |
| Enhancement | TTGGAGCTGGTGGCGTA | SEQ ID |
| Probe | GGCAAGAGTGTTTT | NO: 17 |
| PCR Forward | CTGAAAATGACTGAATA | SEQ ID |
| Primer | TAAACTT | NO: 18 |
| PCR Reverse | TATTGTTGGATCATATT | SEQ ID |
| Primer and | CGTC | NO: 19 |
| Sequencing | | |
| Primer | | |

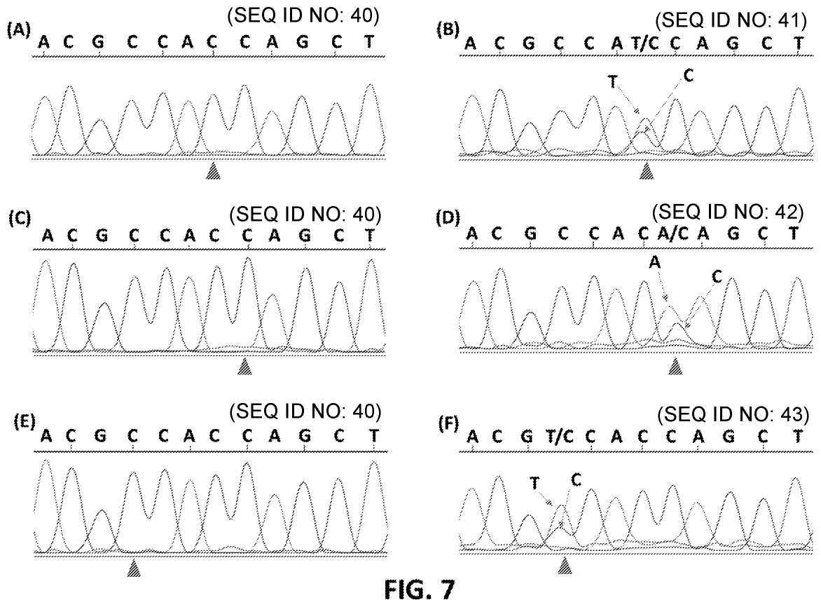

FIG. 7(A) shows the result when we directly sequenced a contrived DNA sample containing G12D mutation without enrichment. This result was used as a reference to determine the degree of enrichment when mutated G12D DNA was enriched by the nucleic acid probe composition, systems, and methods. Experimentally, a contrived DNA sample containing 0.5% mutated G12D DNA was directly PCR-amplified and Sanger-sequenced. The primers used for PCR and Sanger sequencing are also listed in TABLE 1. The wild-type nucleotide at the G12D mutation site is C pointed by the triangle sign. As seen in FIG. 7(A), without enrichment, the G12D mutation could not be detected in a contrived DNA sample containing 0.5% mutated G12D DNA by Sanger sequencing.

FIG. 7(B) shows the result when the capture beads were used to enrich mutated G12D DNA from a contrived DNA sample containing 0.5% mutated G12D DNA in the presence of the enhancement probe. Experimentally, the capture beads containing all seven capture probes, enhancement probe, and a contrived DNA sample were mixed and incubated together for hybridization at room temperature for one hour, in which 2M GuSCN was used as hybridization buffer. After hybridization, magnetic beads were retrieved and then washed with 2×1M NaCl and 1×50 mM NaCl. Thereafter, captured DNA was eluted from capture beads. A portion of eluted DNA was then PCR-amplified and Sanger-sequenced. After enrichment by the nucleic acid probe composition, systems, and methods, the mutated nucleotide T pointed by the triangle sign was clearly seen in FIG. 7(B). In fact, the signal of the mutated nucleotide T at this mutation site is even stronger than that of the wild-type nucleotide C, suggesting that mutated G12D DNA was well enriched, leading to detection of G12D mutation in the contrived DNA sample containing 0.5% mutated G12D DNA by Sanger sequencing.

FIG. 7(C) shows the result when we directly sequenced a contrived DNA sample containing G12C mutation without enrichment. This result was used as a reference to determine the degree of enrichment when mutated G12C DNA was enriched by the nucleic acid probe composition, systems, and methods. Experimentally, the contrived DNA sample containing 0.5% mutated G12C DNA was directly PCR-amplified and Sanger-sequenced. The same primers used for PCR and Sanger sequencing that are listed in TABLE 1 were also used. The wild-type nucleotide at the G12C mutation site is C pointed by the triangle sign. FIG. 7(C) shows that without enrichment, the G12C mutation could not be detected from the contrived DNA sample containing 0.5% mutated G12C DNA by Sanger sequencing.

FIG. 7(D) shows the result when the capture beads containing all seven capture probes were used to enrich mutated G12C DNA from a contrived DNA sample containing 0.5% mutated G12C DNA in the presence of enhancement probe. Experimentally, the capture beads, enhancement probe, and contrived DNA sample were mixed and incubated together for hybridization at room temperature for one hour, in which 2M GuSCN was used as hybridization buffer. After hybridization, magnetic beads were retrieved and then washed with 2×1M NaCl and 1×50 mM NaCl. Thereafter, captured DNA was eluted from magnetic beads. A portion of eluted DNA was then PCR-amplified and Sanger-sequenced. After enrichment by the invented probe composition and method, the mutated nucleotide A pointed by the triangle sign was clearly seen in FIG. 7(D). In fact, the signal of the mutated nucleotide A is even stronger than the signal of the wild-type nucleotide C, suggesting that mutated G12C DNA was well enriched by the nucleic acid probe composition, systems, and methods, leading to detection of G12C mutation from the contrived DNA sample containing 0.5% mutated G12C DNA by Sanger sequencing.

FIG. 7(E) shows the result when we directly sequenced a contrived DNA sample containing G13D mutation without enrichment. This result was used as a reference to determine the degree of enrichment when mutated G13D DNA was enriched by the nucleic acid probe composition, systems, and methods. Experimentally, the contrived DNA sample containing 0.5% mutated G13D DNA was directly PCR amplified and Sanger-sequenced. The wild-type nucleotide at the G13D mutation site is C pointed by the triangle sign. As shown in FIG. 7(E), without enrichment, G13D mutation could not be detected from the contrived DNA sample containing 0.5% mutated G12D DNA by Sanger sequencing.

FIG. 7(F) shows the result when the capture beads were used to enrich mutated G13D from a contrived DNA sample containing 0.5% mutated G13D DNA in the presence of the enhancement probe. Experimentally, the capture beads, enhancement probe, and contrived DNA sample were mixed together for hybridization at room temperature for one hour, in which 2M GuSCN was used as hybridization buffer. After hybridization, magnetic beads were retrieved and then washed with 2×1M NaCl and 1×50 mM NaCl. Thereafter, captured DNA was eluted from magnetic beads. A portion of eluted DNA was then PCR-amplified and Sanger-sequenced. After enrichment by the invented probe composition and method, the mutated nucleotide T pointed by the triangle sign was clearly seen in FIG. 7(F). In fact, the signal of the mutated nucleotide T at the mutation site is even stronger than the signal of the wild-type nucleotide G, suggesting that mutated G13D DNA was well enriched by the nucleic acid probe composition, systems, and methods, leading to detection of G13D mutation from the contrived DNA sample containing 0.5% mutated G13D DNA by Sanger sequencing. It is noted that the nucleic acid probe composition, systems, and methods also produced a similar degree of enrichment of mutated G12A, G12V, G12S, and G12R DNA (data not shown here), leading to detection of these Kras mutations by Sanger sequencing.

### Example 4: Hybridization Buffer Salt Effects

This example is provided to demonstrate that the nucleic acid probe composition works better for enriching and detecting mutated DNA when supplemented with a guanidine-based salt, e.g., guanidinium isothiocyanate (GuSCN), than with salts of conventional hybridization buffers, such as sodium chloride (NaCl), magnesium chloride (MgCl₂), or phosphate buffered saline (PBS), but without a guanidine-based salt. Specifically, we compared four different hybridization buffers for enriching and detecting the Braf (V600E) mutation in a background of wild-type Braf DNA. The contrived DNA sample used in this study was generated by blending 1,200 copies of mutant gBlock Gene Fragment DNA containing a Braf (V600E) mutation and 58,800 copies of wild-type gBlock gene Fragment DNA. The same experimental procedures employed in Example 2 were used in this study except for hybridization buffer.

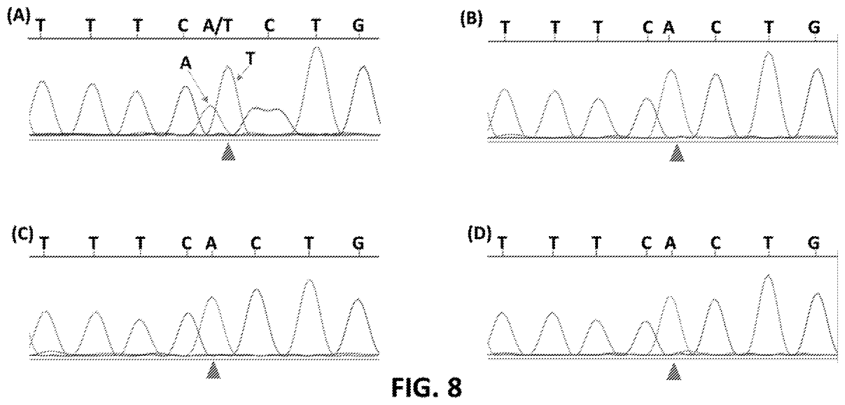

FIG. 8 shows the results of Sanger sequencing generated when four different hybridization buffers were used. Results for FIGS. 8(A), 8(B), 8(C) and 8(D) were obtained with 2M GuSCN, 500 mM NaCl, 10 mM MgCl₂, or 5×PBS buffer, respectively. The wild-type nucleotide in the mutation position was A pointed by the triangle sign, while the mutated nucleotide in this position was T pointed by the triangle sign. As seen from FIG. 8, only 2M GuSCN buffer enriched mutated DNA, leading to detection of mutated Braf (V600E) DNA by Sanger sequencing from a sample containing 2% mutated Braf (V600E) DNA, while the other three buffers failed in adequate enrichment of mutated Braf V600E DNA when the nucleic acid probe composition, systems, and methods were used. We have observed enrichment of mutated DNA when the hybridization buffer containing as little as 0.05 M GuSCN was used though better enrichment was generally achieved with 1-3M GuSCN.

Hybridization buffers containing NaCl, MgCl₂, and PBS, and lacking a guanidine-based salt, are commonly used in hybridizations of nucleic acids. Buffers containing NaCl and MgCl₂, and lacking a guanidine-based salt, have also been used in previous methods involving hybridization of nucleic acid sequences including toehold domains, leading to successful enrichment of mutated DNA in a background of wild-type DNA (see, e.g., Zhang et al., Journal of the American Chemical Society 131:17303-17314 (2009)).

Thus, we were surprised to observe better results for enriching and detecting mutated DNA when supplementing with a guanidine-based salt, again guanidinium isothiocyanate (GuSCN), rather than the NaCl, MgCl₂, and PBS salts of conventional hybridization buffers.

### Example 5: Liquid Phase Hybridization

In all of the above examples, capture probe was pre-immobilized onto magnetic beads before hybridization, meaning that enrichment involves solid-phase hybridization, i.e., a process of forming hybrids between capture probes immobilized on a solid surface and target DNA in a solution. Subsequent separation of magnetic beads from solution, i.e., supernatant, enables enrichment of mutated DNA, and thus detection of mutated DNA in a background of wild-type DNA.

This example is provided to demonstrate the effectiveness of the nucleic acid probe composition, systems, and methods in enrichment of mutated DNA when liquid-phase hybridization, i.e., a process of forming hybrids between capture probes and target DNA in a solution, is involved.

Specifically, we first performed hybridization between capture probes and target DNA molecules in a solution in the presence of the enhancement probe. In this case, the capture probe also contains capture tags that can bind to certain surface molecules of a solid to be captured by the solid such as magnetic beads. After liquid-phase hybridization, the solid is added to the above hybridization system so that the capture probes along with hybrids formed by the capture probes can be isolated by separating the solid from solution, i.e., supernatant, leading to enrichment of mutated DNA.

In this example, we enriched mutated Braf V600E DNA. The capture probe has the following sequence,

(SEQ ID NO: 20)
GGTCAGGTTACCGCTGCGATCGCAGAGATTTCTCTGTAGCTAGACCAA
AATCAAAT,

bold, is a capture sequence that is complementary to mutated DNA containing Braf V600E mutation. The mutation of the mutated DNA corresponds to a single base substitution. The corresponding reverse complement single base of the capture sequence is the T base of the first part that is shown in italics as well as bold. The second part, which is underlined, serves as capture tag, which is complementary to the sequence of an extraction probe that is pre-immobilized onto magnetic beads. Thus, the capture probes along with hybrids formed with them can be isolated by magnetic beads through hybridization between the capture tag of the capture probe and extraction probes pre-immobilized onto magnetic beads. The sequence of the extraction probe pre-immobilized onto magnetic beads is TGCGATCGCAGCGGTAACCTGACC (SEQ ID NO: 21). The third part is a short sequence of AAAT (labeled by italics) at the 3′ end of the capture probe, which was used to prevent the capture probe released from capture beads from being elongated by PCR.

Experimentally, we first mixed and incubated the capture probe, enhancement probe used in Example 2, and a contrived DNA sample containing 2% mutated Braf V600E DNA for hybridization at room temperature, where 2M GuSCN was used as hybridization buffer. Thereafter, the magnetic beads containing the extraction probes were added to the above hybridization system to capture the capture probes and hybrids formed with capture probes by magnetic beads. Then, magnetic beads were retrieved and then washed with 2×1M NaCl and 1×50 mM NaCl. Thereafter, captured DNA was eluted from the magnetic beads. A portion of eluted DNA was then PCR amplified and Sanger-sequenced. The same primers used in Example 2 were used in this study. The contrived DNA samples used were same as those used in Example 4, which contained 2% mutated Braf V600E DNA.

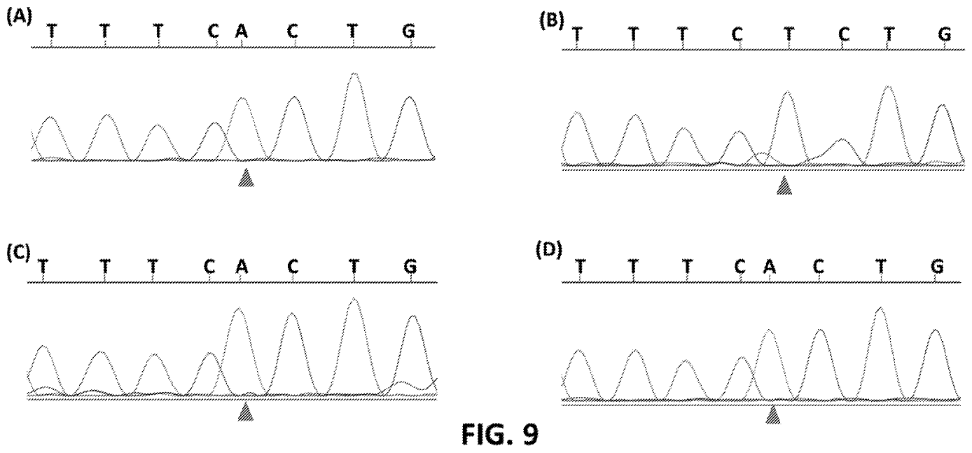

FIG. 9(A) shows the result when we directly sequenced a contrived DNA sample containing 2% mutated Braf V600E DNA without enrichment. This result was used as reference to determine the degree of enrichment when the nucleic acid probe composition, systems, and methods that involved liquid phase hybridization were used to enrich mutated Braf V600E DNA. Again, without enrichment, Braf V600E mutation could not be detected from the sample containing 2% mutated Barf V600E DNA by Sanger sequencing.

FIGS. 9(B), 9(C), and 9(D) shows the results when the nucleic acid probe composition, systems, and methods that involved liquid phase hybridization were used to enrich mutated Braf V600E DNA from the contrived DNA sample containing 2% mutated Braf V600E DNA. The results for FIGS. 9(B), 9(C), and 9(D) were obtained when 2M GuSCN, 500 mM NaCl, and 10 mM MgCl₂ were used as hybridization buffer, respectively. When 2M GuSCN was used as hybridization buffer, the mutated nucleotide T pointed by the triangle sign at the mutation position was clearly seen in FIG. 9(B). In fact, the signal of the mutated nucleotide T at the mutation site is stronger than the signal of the wild-type nucleotide A, suggesting that mutated Braf V600E DNA was significantly enriched, thus becoming detectable by Sanger sequencing.

This result shows that the nucleic acid probe composition, systems, and methods also work well for enrichment of mutated DNA when liquid phase hybridization is involved.

Also, as seen from comparing FIG. 9(B) with FIG. 9(C) and FIG. 9(D), the nucleic acid probe composition, systems, and methods involving liquid phase hybridization again provided better results for enriching and detecting mutated DNA when supplementing with a guanidine-based salt, again guanidinium isothiocyanate (GuSCN), rather than the NaCl or MgCl₂ salts of conventional hybridization buffers, consistent with our previous results involving hybridization buffer salt effects.

### Example 6: Recovery of Mutated DNA

This example is provided to demonstrate that the invented probe composition and method can concurrently extract and enrich mutated DNA from a sample containing mutated DNA with a high recovery and a high degree of enrichment. Another problem of previous methods such as the method of Jeffreys et al. (2003) is that only a small percentage of mutated DNA can be recovered during the enrichment process. We found that the nucleic acid probe composition, systems, and methods could achieve a high recovery of mutated DNA while still maintaining a high degree of enrichment.

In this example, we employed the experimental procedure used in Example 2 to enrich and recover mutated Braf V600E DNA from the contrived DNA samples containing 2% mutated Braf V600E DNA. After extraction and enrichment, a portion of extracted DNA was subject to Sanger sequencing as in Example 2, while another portion of extracted DNA was subject to qPCR, which was performed in LUNA Universal qPCR Master Mix (New England Biolabs), to estimate the recovery of mutated Braf V600E DNA. The same pair of primers used in Example 2 was used in qPCR as well. The same experiment was also repeated five times to assess the reproducibility of the nucleic acid probe composition, systems, and methods. TABLE 2 shows the degree of enrichment and the recovery rate for each repeat. The degree of enrichment is estimated based on the ratio of the peak height of the mutated nucleotide to that of the wild-type nucleotide, from which we also estimated the percentage of wild-type DNA in extracted DNA. Since our qPCR could not distinguish mutated DNA from wild-type DNA, the recovery listed in TABLE 2 was determined by subtracting the estimated amount of wild-type DNA in extracted DNA. The averaged degrees of enrichment and recovery over five repeats were 2.8 and 65%, respectively, suggesting that the nucleic acid probe composition, systems, and methods can be utilized for concurrent extraction and enrichment of mutated DNA from a sample containing mutated DNA.

TABLE 2
Degree of enrichment and recovery of mutated Braf V600E DNA

| | Degree of | Recovery |
|---|---|---|
| Experiment | Enrichment | (%) |
| Repeat 1 | 2.4 | 60 |
| Repeat 2 | 2.1 | 64 |
| Repeat 3 | 3 | 67 |
| Repeat 4 | 2.6 | 65 |
| Repeat 5 | 4 | 71 |

### Example 7: Enrichment of Mutated G12D Kras DNA by 1,000 Fold

This example is provided to demonstrate that the nucleic acid probe composition, systems, and methods could enrich mutated DNA by at least 1,000 fold. In addition, this example is also provided to demonstrate that the nucleic acid probe composition, systems, and methods are effective when the toehold subsequence of the capture probe is either in the 5′-end or 3′-end position. In Example 3, the toehold subsequence of the capture probe used was in the 5′-end position, while the toehold subsequence of the enhancement probe was in the 3′-end position. In the present example, in which we enriched mutated G12D Kras DNA, the toehold subsequence of the capture probe is in the 3′-end position

(SEQ ID NO: 22)
(TTGGAGCTGATGGCGTAGGCAAGAGTGCAAAA),

while the toehold subsequence of the enhancement probe is in the 5′-end position

(SEQ ID NO: 23)
(GTGGTAGTTGGAGCTGGTGGCGTAGGCTTTT).

The sequences shown in italics in both probes are the respective toehold subsequences. The short sequences of TTTT and AAAA shown in bold are used to prevent the capture and enhancement probes from being elongated by PCR, as these TTTT and AAAA sequences are not complementary to the target sequences.

We used a “G12D” gBlock Gene Fragment to mimic mutated G12D Kras DNA and a “wild-type” gBlock Gene Fragment to mimic wild-type DNA. Each contrived DNA sample containing mutated G12D Kras DNA was generated by mixing “G12D” gBlock Gene Fragment and “wild-type” gBlock Gene Fragment at a ratio of 1/1000, i.e., 0.1%, of mutant to wild-type DNA. The primers used for PCR and Sanger sequencing were the same as those listed in TABLE 1. In this study, we only immobilized the capture probes that are specific to mutated G12D Kras DNA onto magnetic beads to make capture beads. The rest of the experimental conditions are similar to those used in Example 3.

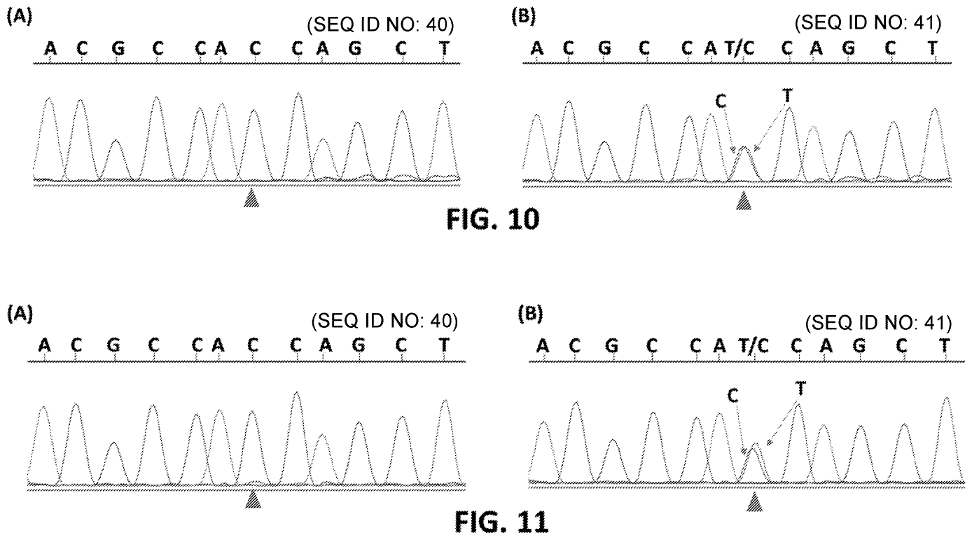

FIG. 10(A) shows the result when no enhancement probes are present, where no mutated G12D was detected, while FIG. 10(B) shows the result when enhancement probes are present. After enrichment by this new probe system, the mutated nucleotide T pointed by the triangle sign was clearly seen in FIG. 10(B). In fact, the signal of the mutated nucleotide T at this mutation site is nearly same as that of the wild-type nucleotide C, suggesting that mutated G12D DNA was enriched by 1,000 fold by toehold exchange.

### Example 8: Sequences of Mixing Capture Probes, Enhancement Probes, and DNA Samples

This example is provided to demonstrate that the nucleic acid probe composition, systems, and methods still work well when the sequence, i.e., order, of mixing the capture probes (beads), enhancement probes, and DNA samples is varied. In the above examples, the capture probes (beads), enhancement probes, and DNA samples are mixed together concurrently for hybridization and toehold displacement/toehold exchange. In the present example, the capture probes (beads) and DNA samples were first mixed together for incubation, followed by adding enhancement probes to the mixture for additional incubation. In this example, we used the same probes used in Example 7 to enrich mutated G12D Kras DNA. Briefly, the contrived DNA samples containing mutated G12D Kras DNA were generated by mixing “G12D” gBlock Gene Fragment and “wild-type” gBlock Gene Fragment at a ratio of 1/200, i.e., 0.5%, of mutant to wild-type DNA. In this study, we only immobilized the capture probes that are specific to mutated G12D Kras DNA onto magnetic beads to make capture beads. The primers used for PCR and Sanger sequencing were the same as those listed in TABLE 1.

Results for FIG. 11(A) were obtained by mixing the capture probes (beads) and contrived DNA sample, which contained 0.5% of mutated G12D DNA, together for 60 min for hybridization in the absence of enhancement probes. Then, the capture beads were isolated and washed. Finally, extracted DNA was eluted from the beads and subjected to Sanger sequencing. As seen in FIG. 11(A), no mutated G12D was detected.

Results for FIG. 11(B) were obtained by mixing the capture probes (beads) and same contrived DNA sample together for 60 min for hybridization, followed by adding enhancement probes to the mixture for incubation for another 30 min. Then, the capture beads were isolated and washed. Finally, extracted DNA was eluted from the beads and subjected sanger sequencing. The mutated nucleotide T pointed by the triangle sign was clearly seen in FIG. 11(B) and the signal of the mutated nucleotide T at the mutation site is nearly same as that of the wild-type nucleotide C. This example clearly indicates that the nucleic acid probe composition, systems, and methods work well when the sequence, i.e., order, of mixing the capture probes (beads), enhancement probes, and DNA samples is varied.

### Example 9: Mutation Analysis of Formalin-Fixed Paraffin-Embedded Tumor Tissues

Mutation analysis of formalin-fixed paraffin-embedded (“FFPE”) tumor tissues, which reveals if relevant mutations are present in tumors, is essential to assessment of patients' eligibility for a targeted therapy, and thus is an example of mutation analysis of biologically relevant samples.

This example is provided to show that the nucleic acid probe composition, systems, and methods are compatible with FFPE samples, leading to enrichment of mutated DNA from FFPE samples, and thus can be used for mutation analysis of these biologically relevant samples.

The FFPE tumor tissue sample used in this example contains 5% mutated Kras G12D mutation. Therefore, we first used the nucleic acid probe composition, systems, and methods to extract/enrich mutated DNA containing the Kras G12D mutation from the FFPE sample, followed by Sanger sequencing of extracted DNA to determine whether mutated Kras G12D DNA is present in this FFPE sample.

The sequence of the capture probe and the enhancement probe used are

(SEQ ID NO: 22)
TTGGAGCTGATGGCGTAGGCAAGAGTGCAAAA
and
(SEQ ID NO: 23)
GTGGTAGTTGGAGCTGGTGGCGTAGGCTTTT,

respectively.

The sequences underlined in both the capture probe and the enhancement probe are the variant target domain and the reference target domain, respectively. The sequences shown in italics in both probes are the toehold subsequences. The short sequences of TTTT and AAAA shown in bold are used to prevent the capture and enhancement probes from being elongated by PCR. The capture probe was pre-immobilized onto magnetic beads to make capture beads. The same primers listed in TABLE 1 were used in PCR and Sanger sequencing.

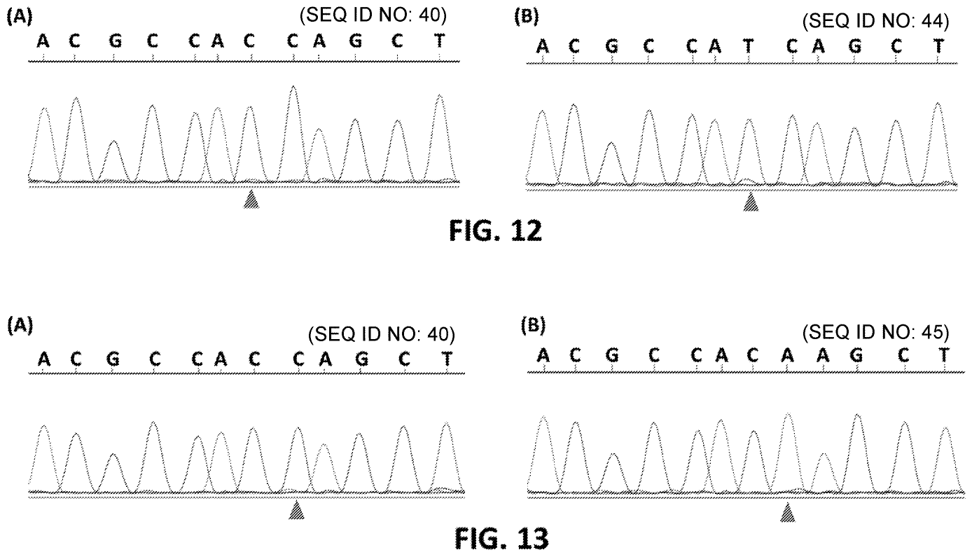

FIG. 12 shows the result of Sanger sequencing of extracted DNA from this FFPE sample without enrichment by toehold displacement/toehold exchange (A) and with enrichment by toehold displacement/toehold exchange (B). As shown in FIG. 12, since we were sequencing the antisense strand, the wild-type and mutated nucleotides at the mutation site are C pointed by the triangle sign and T pointed by the triangle sign, respectively. Clearly, without enrichment, mutated Kras G12D DNA could not be detected from the FFPE sample (FIG. 12(A)), while it was clearly detected when the invented nucleic acid probe composition, systems, and methods were used to enrich/extract mutated DNA from the same FFPE sample (FIG. 12(B)).

This example demonstrated that the nucleic acid probe composition, systems, and methods are compatible with FFPE samples, meaning that they are effective when used to enrich/extract mutated DNA from FFPE samples.

### Example 10: Mutation Analysis of Serum Samples

Mutation analysis of serum samples is another example of mutation analysis in biologically relevant samples.

This example is provided to demonstrate that the nucleic acid probe composition, systems, and methods are compatible with serum, allowing enrichment mutated DNA from serum, and thus can be used for mutation analysis of these biologically relevant samples.

The serum sample used in this example was collected from a lung cancer patient and the tumor tissue analysis had revealed that the tumor of this lung cancer patient contained the Kras G12C mutation. Therefore, we first used the nucleic acid probe composition, systems, and methods to extract/enrich mutated DNA containing the Kras G12C mutation from the serum sample, followed by Sanger sequencing of extracted DNA to find out if the mutated Kras G12C DNA is present in this serum sample. The sequences of the capture probe and the enhancement probe used are

(SEQ ID NO: 24)
TTGGAGCTTGTGGCGTAGGCAAGAGTGCAAAA
and
(SEQ ID NO: 23)
GTGGTAGTTGGAGCTGGTGGCGTAGGCTTTT,

respectively.

The sequences underlined in both the capture probe and the enhancement probe are the variant target domain and the reference target domain, respectively. The sequences shown in italics in both probes are the toehold subsequences. The short sequences of TTTT and AAAA shown in bold are used to prevent the capture and enhancement probes from being elongated by PCR. The capture probe was pre-immobilized onto magnetic beads to make capture beads. The same primers listed in TABLE 1 were used in PCR and Sanger sequencing.

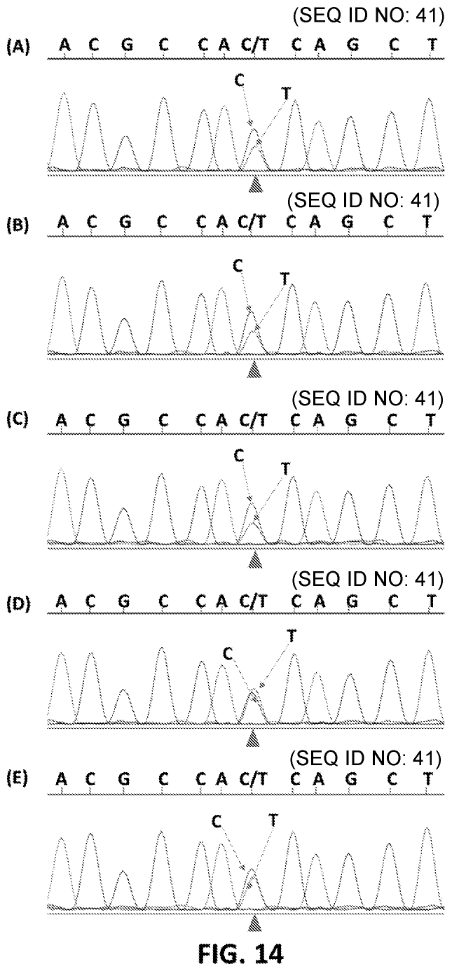

FIG. 13 shows the result of Sanger sequencing of extracted DNA from this serum sample without enrichment by toehold exchange (A) and with enrichment by toehold exchange (B). As shown in FIG. 13, since we were sequencing the antisense strand, the wild-type and mutated nucleotides at the mutation site are C pointed by the triangle sign and A pointed by the triangle sign, respectively. Clearly, without enrichment, mutated Kras G12C DNA could not be detected from the serum sample (FIG. 13(A)), while it was clearly detected when the invented nucleic acid probe composition, systems, and methods were used to enrich/extract mutated DNA from the same serum sample (FIG. 13(B)).

This example demonstrated that the nucleic acid probe composition, systems, and methods are compatible with serum, meaning they are effective when used to enrich/extract mutated DNA from serum.

### Example 11: Capture and Enhancement Probes Containing Mismatched Nucleotides

This example demonstrates that the invented probe composition, system, and method for enriching, extracting, and detecting a variant target nucleic acid in a sample comprising the variant target nucleic acid and a reference target nucleic acid work well even when the capture probe and/or enhancement probes comprise one or more mismatched nucleotides. A mismatched nucleotide refers to a nucleotide in the capture probe or enhancement probe that is not complementary to the corresponding nucleotide of the target nucleic acids, and is in a nucleotide position other than that where the insertion, deletion or substitution occurs.

We first studied if the invented probe composition, system, and method are still effective when the enhancement probe comprises a mismatched nucleotide, while the capture probe comprises no mismatched nucleotide. In this case, we enriched mutated G12D Kras DNA, where the sequence of the capture probe is TTGGAGCTGATGGCGTAGGCAAGAGTGCAAAA (SEQ ID NO: 22), and the sequence of the enhancement probe that comprises a mismatched nucleotide is GTGGTAGATGGAGCTGGTGGCGTAGGCTTTT (SEQ ID NO: 31), GTGGTAGCTGGAGCTGGTGGCGTAGGCTTTT (SEQ ID NO: 32) or GTGGTAGGTGGAGCTGGTGGCGTAGGCTTTT (SEQ ID NO: 33). The nucleotide A/C/G shown in bold is the mismatched nucleotide. The short sequences of TTTT and AAAA underlined are used to prevent the capture and enhancement probes from being elongated by PCR, as these sequences are not complementary to the target sequences. Experimentally, we used a “G12D” gBlock Gene Fragment to mimic mutated G12D Kras DNA and a “wild-type” gBlock Gene Fragment to mimic wild-type DNA. Each contrived DNA sample containing mutated G12D Kras DNA was generated by mixing “G12D” gBlock Gene Fragment and “wild-type” gBlock Gene Fragment at a ratio of 1/50, i.e., 2% of mutant to wild-type DNA. The primers used for PCR and Sanger sequencing were the same as those listed in TABLE 1. The rest of the experimental conditions are similar to those used in Example 7. FIGS. 14(A), (B), and (C) show the result when the enhancement probe comprising a mismatched nucleotide of A, C, or G were used in enrichment. It is seen that the mutated nucleotide T pointed by arrow was clearly seen in FIGS. 14(A), (B) and (C), suggesting successful enrichment of mutated G12D DNA by the invented probe composition, system, and method when the enhancement probe used comprises a mismatched nucleotide. We have also studied if the invented probe composition, system, and method is effective when the capture probe comprises a mismatched nucleotide while the enhancement probe has no mismatched nucleotide. In this case, successful enrichment of mutant DNA by the invented probe composition, system, and method is also achieved. Moreover, we have observed successful enrichment of mutant DNA by the invented probe composition, system, and method when both the capture and enhancement probes comprise a mismatched nucleotide.

We next studied whether the invented probe composition, system, and method are effective when the enhancement probe comprises multiple mismatched nucleotides. We have studied enrichment of mutant DNA by the invented probe composition, system, and method when both the capture and enhancement probes comprise as many as five (5) mismatched nucleotides. FIG. 14(D) shows the result when both the capture and enhancement probes comprise three (3) mismatched nucleotides. The sequence of the capture and enhancement probe is TAGGTGCTGATGGCGTATGCAAGAGTGCAAAA (SEQ ID NO: 34) or TGGTAGTAGGTGCTGGTGGCGTATGCTTTT (SEQ ID NO: 35), respectively.

The nucleotide shown in bold is the mismatched nucleotide. The short sequences of TTTT and AAAA underlined are used to prevent the capture and enhancement probes from being elongated by PCR, as these sequences are not complementary to the target sequences. Experimentally, we used a “G12D” gBlock Gene Fragment to mimic mutated G12D Kras DNA and a “wild-type” gBlock Gene Fragment to mimic wild-type DNA. Each contrived DNA sample containing mutated G12D Kras DNA was generated by mixing “G12D” gBlock Gene Fragment and “wild-type” gBlock Gene Fragment at a ratio of 1/50, i.e., 2% of mutant to wild-type DNA. The primers used for PCR and Sanger sequencing were the same as those listed in TABLE 1. The rest of the experimental conditions are similar to those used in Example 7. It is seen that the mutated nucleotide T pointed by arrow was clearly seen in FIG. 14(D), suggesting successful enrichment of mutated G12D DNA by the invented probe composition, system, and method when both the capture and enhancement probes used comprises three (3) mismatched nucleotides.

FIG. 14(E) shows the result when both the capture and enhancement probes comprise five (5) mismatched nucleotides. The sequence of the capture and enhancement probe is TAGGTGCCGATGGCGAATGCAAGAGTGCAAAA (SEQ ID NO: 36) or TGGTAGTAGGTGCCGGTGGCGAATGCTTTT (SEQ ID NO: 37), respectively. The nucleotide shown in bold is the mismatched nucleotide. The short sequences of TTTT and AAAA underlined are used to prevent the capture and enhancement probes from being elongated by PCR, as these sequences are not complementary to the target sequences. Experimentally, we used a “G12D” gBlock Gene Fragment to mimic mutated G12D Kras DNA and a “wild-type” gBlock Gene Fragment to mimic wild-type DNA. Each contrived DNA sample containing mutated G12D Kras DNA was generated by mixing “G12D” gBlock Gene Fragment and “wild-type” gBlock Gene Fragment at a ratio of 1/50, i.e., 2% of mutant to wild-type DNA. The primers used for PCR and Sanger sequencing were the same as those listed in TABLE 1. The rest of the experimental conditions are similar to those used in Example 7. It is seen that the mutated nucleotide T pointed by arrow was clearly seen in FIG. 14(D), suggesting successful enrichment of mutated G12D DNA by the invented probe composition, system, and method when both the capture and enhancement probes used comprise five (5) mismatched nucleotides.

### CLAUSES

1. A nucleic acid probe composition for enriching, extracting, and detecting a variant target nucleic acid in a sample comprising the variant target nucleic acid and a reference target nucleic acid, wherein the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of one to 100 nucleotides, or a combination thereof, relative to the nucleic acid sequence of the nucleic acid strand segment of the reference target nucleic acid, the composition comprising:
(a) a capture probe comprising a single-stranded oligonucleotide that has a 5′ end and a 3′ end and comprises a variant target domain and either a 5′ upstream toehold domain or a 3′ downstream toehold domain, but not both, contiguous to the variant target domain; and
(b) an enhancement probe comprising a single-stranded oligonucleotide that has a 5′ end and a 3′ end and comprises a reference target domain and either a 3′ downstream toehold domain or a 5′ upstream toehold domain, but not both, contiguous to the reference target domain, wherein
(i) the 5′ upstream toehold domain has a nucleic acid sequence that is a reverse complement of 3′ subsequences of the nucleic acid sequences of the strand segments of the variant and reference target nucleic acids extending from the 3′ ends of the strand segments of the variant and reference target nucleic acids;
(ii) the 3′ downstream toehold domain has a nucleic acid sequence that is a reverse complement of 5′ subsequences of the nucleic acid sequences of the strand segments of the variant and reference target nucleic acids extending from the 5′ ends of the strand segments of the variant and reference target nucleic acids;
(iii) the variant target domain has a nucleic acid sequence that is a reverse complement of an intermediate subsequence of the strand segment of the variant target nucleic acid that extends contiguously between the 5′ and 3′ subsequences of the strand segment of the variant target nucleic acid and comprises the substitution, insertion, or deletion, or combination thereof;
(iv) the reference target domain has a nucleic acid sequence that is a reverse complement of an intermediate subsequence of the strand segment of the reference target nucleic acid that extends contiguously between the 5′ and 3′ subsequences of the strand segment of the reference target nucleic acid and does not comprise the substitution, insertion, or deletion, or combination thereof;
(v) if the capture probe comprises the 5′ upstream toehold domain, then the enhancement probe comprises the 3′ downstream toehold domain, whereas if the capture probe comprises the 3′ downstream toehold domain, then the enhancement probe comprises the 5′ upstream toehold domain;
(vi) the nucleic acid sequences of the 5′ upstream toehold domain and the 3′ downstream toehold domain each comprise 2 to 100 nucleotides; and
(vii) the composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

2. The composition of clause 1, wherein:
the single-stranded oligonucleotide of the capture probe comprises one or more of DNA, RNA, locked nucleic acids, or peptide nucleic acids, or a combination thereof; and
the single-stranded oligonucleotide of the enhancement probe comprises one or more of DNA, RNA, locked nucleic acids, or peptide nucleic acids, or a combination thereof.

3. The composition of clause 1 or clause 2, wherein the substitution, insertion, or deletion, or combination thereof, comprises one or more of a single-base substitution, a multiple-base substitution, a single-base insertion, a multiple-base insertion, a single-base deletion, or a multiple-base deletion.

4. The composition of any one of clauses 1-3, further comprising a guanidine-based salt.

5. The composition of clause 4, wherein the guanidine-based salt comprises one or more of guanidine or a guanidine derivative with the structure (R1R2N)(R3R4N)C═N—R5, wherein R1, R2, R3, R4, and R5 each independently are either (i) hydrogen or (ii) a functional group comprising 1 to 4 carbons with hydrogens, with the proviso that the sum of carbons in R1 to R5 is less than or equal to 15.

6. The composition of clause 4 or clause 5, wherein the composition is aqueous and the guanidine-based salt is present in the composition at a concentration of 0.05 to 8 M.

7. The composition of any one of clauses 1-6, wherein the nucleic acid sequences of the strand segments of the variant and reference target nucleic acids each comprise 15 to 400 nucleotides.

8. The composition of any one of clauses 1-7, wherein the variant target domain and the reference target domain each comprise 1 to 200 nucleotides.

9. The composition of any one of clauses 1-7, wherein the capture probe comprises only one capture probe and the enhancement probe comprises only one enhancement probe, wherein the capture probe is specific to a single variant target nucleic acid, and the enhancement probe is specific to a single reference target nucleic acid.

10. The composition of any one of clauses 1-7, wherein the capture probe comprises a plurality of capture probes, and the enhancement probe comprises only one enhancement probe, wherein each of the capture probes is specific to a different variant target nucleic acid, and the enhancement probe is specific to a single reference target nucleic acid.

11. The composition of any one of clauses 1-7, wherein the capture probe comprises a plurality of capture probes, and the enhancement probe comprises a plurality of enhancement probes, wherein each of the capture probes is specific to a different variant target nucleic acid, and each of the enhancement probes is specific to a different reference target nucleic acid, wherein each of the reference target nucleic acids is a reference nucleic acid to one or more of the variant target nucleic acids.

12. The composition of any one of clauses 1-11, wherein the composition does not comprise the variant or reference target nucleic acids.

13. The composition of any one of clauses 1-11, wherein the composition comprises one or more of the variant and/or reference target nucleic acids.

14. The composition of any one of clauses 1-13, wherein the capture probe comprises at least 6 pairs of adjacent matched nucleotides with respect to the nucleic acid sequence of the strand segment of the variant target nucleic acid, and the enhancement probe comprises at least 6 pairs of adjacent matched nucleotides with respect to the nucleic acid sequence of the strand segment of the reference target nucleic acid.

15. The composition of any one of clauses 1-13, wherein the capture probe contains only matched nucleotides with respect to the nucleic acid sequence of the strand segment of the variant target nucleic acid, and the enhancement probe contains only matched nucleotides with respect to the nucleic acid sequence of the strand segment of the reference target nucleic acid.

16. The composition of any one of clauses 1-15, wherein the composition does not comprise any other oligonucleotides that are complementary to the 5′ subsequences, the intermediate subsequences, or the 3′ subsequences of the strand segments of the variant or reference target nucleic acids and does not comprise any other nucleic acids that are complementary to the capture probe or the enhancement probe.

17. A system for enriching, extracting, and detecting a variant target nucleic acid in a sample comprising the variant target nucleic acid and a reference target nucleic acid, wherein the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid, the system comprising:
(a) the composition of any one of clauses 1-16; and
(b) a solid, wherein the capture probe is immobilized on a surface of the solid.

18. A system for enriching, extracting, and detecting a variant target nucleic acid in a sample comprising the variant target nucleic acid and a reference target nucleic acid, wherein the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid, the system comprising:
(a) the composition of any one of clauses 1-16; and
(b) a solid, wherein:
(i) the capture probe comprises at least one binding tag; and
(ii) the solid comprises an extraction tag that binds to the at least one binding tag.

19. A method for enriching, extracting, and detecting a variant target nucleic acid in a sample comprising the variant target nucleic acid and a reference target nucleic acid, wherein the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid, the method comprising steps of:
(1) combining the sample, a capture probe, and an enhancement probe, thereby forming a hybridization composition;
(2) incubating the hybridization composition at a temperature that promotes the formation of hybrids of the capture probe and the variant target nucleic acid and hybrids of the enhancement probe and the reference target nucleic acid by toehold displacement and/or toehold exchange;
(3) isolating the hybrids formed by the capture probe from the hybrids formed by the enhancement probe;
(4) eluting the variant target nucleic acid from the hybrids of the capture probe and the variant target nucleic acid; and
(5) detecting the variant target nucleic acid, wherein:
(a) the capture probe comprises a single-stranded oligonucleotide that has a 5′ end and a 3′ end and comprises a variant target domain and either a 5′ upstream toehold domain or a 3′ downstream toehold domain, but not both, contiguous to the variant target domain; and
(b) the enhancement probe comprises a single-stranded oligonucleotide that has a 5′ end and a 3′ end and comprises a reference target domain and either a 3′ downstream toehold domain or a 5′ upstream toehold domain, but not both, contiguous to the reference target domain, wherein
(i) the 5′ upstream toehold domain has a nucleic acid sequence that is a reverse complement of 3′ subsequences of the nucleic acid sequences of the strand segments of the variant and reference target nucleic acids extending from the 3′ ends of the strand segments of the variant and reference target nucleic acids;
(ii) the 3′ downstream toehold domain has a nucleic acid sequence that is a reverse complement of 5′ subsequences of the nucleic acid sequences of the strand segments of the variant and reference target nucleic acids extending from the 5′ ends of the strand segments of the variant and reference target nucleic acids;
(iii) the variant target domain has a nucleic acid sequence that is a reverse complement of an intermediate subsequence of the strand segment of the variant target nucleic acid that extends contiguously between the 5′ and 3′ subsequences of the strand segment of the variant target nucleic acid and comprises the substitution, insertion, or deletion, or combination thereof;
(iv) the reference target domain has a nucleic acid sequence that is a reverse complement of an intermediate subsequence of the strand segment of the reference target nucleic acid that extends contiguously between the 5′ and 3′ subsequences of the strand segment of the reference target nucleic acid and does not comprise the substitution, insertion, or deletion, or combination thereof;
(v) if the capture probe comprises the 5′ upstream toehold domain, then the enhancement probe comprises the 3′ downstream toehold domain, whereas if the capture probe comprises the 3′ downstream toehold domain, then the enhancement probe comprises the 5′ upstream toehold domain;
(vi) the nucleic acid sequences of the 5′ upstream toehold domain and the 3′ downstream toehold domain each comprise 2 to 100 nucleotides; and
(vii) the hybridization composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

20. The method of clause 19, wherein the method further comprises a step (0) of combining the sample and the capture probe, thereby forming an initial sample-capture probe composition prior to step (1).

21. The method of clause 19, wherein the method further comprises a step (0) of combining the sample and the enhancement probe, thereby forming an initial sample-enhancement probe composition prior to step (1).

22. The method of clause 19, wherein:
(i) the method further comprises a step (0) of immobilizing the capture probe on a surface of a solid prior to step (1); and
(ii) step (3) comprises isolating the hybrids formed by the capture probe from the hybrids formed by the enhancement probe by separating the solid from the hybridization composition.

23. The method of clause 19, wherein:
(i) the capture probe comprises at least one binding tag; and
(ii) step (3) comprises isolating the hybrids formed by the capture probe from the hybrids formed by the enhancement probe by contacting the hybridization solution with a solid that comprises an extraction tag that binds to the at least one binding tag, then separating the solid from the hybridization composition.

24. The method of any one of clauses 19-23, wherein step (5) comprises detecting the variant target nucleic acid by a DNA analysis method comprising one or more of real-time PCR, microarray, fragment size analysis, Sanger sequencing, next-generation sequencing, or digital PCR, or a combination thereof.

25. The method of any one of clauses 19-24, wherein:
the oligonucleotide of the capture probe comprises one or more of DNA, RNA, locked nucleic acids, or peptide nucleic acids, or a combination thereof; and
the oligonucleotide of the enhancement probe comprises one or more of DNA, RNA, locked nucleic acids, or peptide nucleic acids, or a combination thereof.

26. The method of any one of clauses 19-25, wherein the substitution, insertion, or deletion comprises one or more of a single-base substitution, a multiple-base substitution, a single-base insertion, a multiple-base insertion, a single-base deletion, or a multiple-base deletion.

27. The method of any one of clauses 19-26, wherein the hybridization solution further comprises a guanidine-based salt.

28. The method of clause 27, wherein the guanidine-based salt comprises one or more of guanidine or a guanidine derivative with the structure (R1R2N)(R3R4N)C═N—R5, wherein R1, R2, R3, R4, and R5 each independently are either (i) hydrogen or (ii) a functional group comprising 1 to 4 carbons with hydrogens, with the proviso that the sum of carbons in R1 to R5 is less than or equal to 15.

29. The method of clause 27 or clause 28, wherein the guanidine-based salt is present in the hybridization composition at a concentration of 0.05 to 8 M.

30. The method of any one of clauses 19-29, wherein the nucleic acid sequences of the strand segments of the variant and reference target nucleic acids each comprise 15 to 400 nucleotides.

31. The method of any one of clauses 19-30, wherein the variant target domain and the reference target domain each comprise 1 to 200 nucleotides.

32. The method of any one of clauses 19-31, wherein the capture probe comprises only one capture probe and the enhancement probe comprises only one enhancement probe, wherein the capture probe is specific to a single variant target nucleic acid, and the enhancement probe is specific to a single reference target nucleic acid.

33. The method of any one of clauses 19-31, wherein the capture probe comprises a plurality of capture probes, and the enhancement probe comprises only one enhancement probe, wherein each of the capture probes is specific to a different variant target nucleic acid, and the enhancement probe is specific to a single reference target nucleic acid.

34. The method of any one of clauses 19-31, wherein the capture probe comprises a plurality of capture probes, and the enhancement probe comprises a plurality of enhancement probes, wherein each of the capture probes is specific to a different variant target nucleic acid, and each of the enhancement probes is specific to a different reference target nucleic acid, wherein each of the reference target nucleic acids is a reference nucleic acid to one or more of the variant target nucleic acids.

35. The method of any one of clauses 19-34, wherein the capture probe comprises at least 6 pairs of adjacent matched nucleotides with respect to the nucleic acid sequence of the strand segment of the variant target nucleic acid, and the enhancement probe comprises at least 6 pairs of adjacent matched nucleotides with respect to the nucleic acid sequence of the strand segment of the reference target nucleic acid.

36. The method of any one of clauses 19-34, wherein the capture probe contains only matched nucleotides with respect to the nucleic acid sequence of the strand segment of the variant target nucleic acid, and the enhancement probe contains only matched nucleotides with respect to the nucleic acid sequence of the strand segment of the reference target nucleic acid.

37. The method of any one of clauses 19-36, wherein the hybridization composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

### REFERENCE TO A “SEQUENCE LISTING” SUBMITTED AS AN XML FILE

The material in the XML file, named “GLC-65843WO-Sequence-Listing.xml”, created Aug. 12, 2023, file size of 45,056 bytes, is hereby incorporated by reference.

1. A nucleic acid probe composition for enriching, extracting, and detecting a variant target nucleic acid in a sample comprising the variant target nucleic acid and a reference target nucleic acid, wherein the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of one to 100 nucleotides, or a combination thereof, relative to the nucleic acid sequence of the nucleic acid strand segment of the reference target nucleic acid, the composition comprising:
(a) a capture probe comprising a single-stranded oligonucleotide that has a 5′ end and a 3′ end and comprises a variant target domain and either a 5′ upstream toehold domain or a 3′ downstream toehold domain, but not both, contiguous to the variant target domain; and
(b) an enhancement probe comprising a single-stranded oligonucleotide that has a 5′ end and a 3′ end and comprises a reference target domain and either a 3′ downstream toehold domain or a 5′ upstream toehold domain, but not both, contiguous to the reference target domain, wherein
(i) the 5′ upstream toehold domain has a nucleic acid sequence that is a reverse complement of 3′ subsequences of the nucleic acid sequences of the strand segments of the variant and reference target nucleic acids extending from the 3′ ends of the strand segments of the variant and reference target nucleic acids;
(ii) the 3′ downstream toehold domain has a nucleic acid sequence that is a reverse complement of 5′ subsequences of the nucleic acid sequences of the strand segments of the variant and reference target nucleic acids extending from the 5′ ends of the strand segments of the variant and reference target nucleic acids;
(iii) the variant target domain has a nucleic acid sequence that is a reverse complement of an intermediate subsequence of the strand segment of the variant target nucleic acid that extends contiguously between the 5′ and 3′ subsequences of the strand segment of the variant target nucleic acid and comprises the substitution, insertion, or deletion, or combination thereof;
(iv) the reference target domain has a nucleic acid sequence that is a reverse complement of an intermediate subsequence of the strand segment of the reference target nucleic acid that extends contiguously between the 5′ and 3′ subsequences of the strand segment of the reference target nucleic acid and does not comprise the substitution, insertion, or deletion, or combination thereof;
(v) if the capture probe comprises the 5′ upstream toehold domain, then the enhancement probe comprises the 3′ downstream toehold domain, whereas if the capture probe comprises the 3′ downstream toehold domain, then the enhancement probe comprises the 5′ upstream toehold domain;
(vi) the nucleic acid sequences of the 5′ upstream toehold domain and the 3′ downstream toehold domain each comprise 2 to 100 nucleotides; and
(vii) the composition does not require any other oligonucleotides that are complementary to the 5′ subsequences, the intermediate subsequences, or the 3′ subsequences of the strand segments of the variant or reference target nucleic acids and does not require any other nucleic acids that are complementary to the capture probe or the enhancement probe.

2. The composition of claim 1, wherein:
the single-stranded oligonucleotide of the capture probe comprises one or more of DNA, RNA, locked nucleic acids, or peptide nucleic acids, or a combination thereof; and
the single-stranded oligonucleotide of the enhancement probe comprises one or more of DNA, RNA, locked nucleic acids, or peptide nucleic acids, or a combination thereof.

3. The composition of claim 1, wherein the substitution, insertion, or deletion, or combination thereof, comprises one or more of a single-base substitution, a multiple-base substitution, a single-base insertion, a multiple-base insertion, a single-base deletion, or a multiple-base deletion.

4. The composition of claim 1, further comprising a guanidine-based salt.

5. The composition of claim 4, wherein the guanidine-based salt comprises one or more of guanidine or a guanidine derivative with the structure (R1R2N)(R3R4N)C═N—R5, wherein R1, R2, R3, R4, and R5 each independently are either (i) hydrogen or (ii) a functional group comprising 1 to 4 carbons with hydrogens, with the proviso that the sum of carbons in R1 to R5 is less than or equal to 15.

6. The composition of claim 4, wherein the composition is aqueous and the guanidine-based salt is present in the composition at a concentration of 0.05 to 8 M.

7. The composition of claim 1, wherein the nucleic acid sequences of the strand segments of the variant and reference target nucleic acids each comprise 15 to 400 nucleotides.

8. The composition of claim 1, wherein the variant target domain and the reference target domain each comprise 1 to 200 nucleotides.

9. The composition of claim 1, wherein the capture probe comprises only one capture probe and the enhancement probe comprises only one enhancement probe, wherein the capture probe is specific to a single variant target nucleic acid, and the enhancement probe is specific to a single reference target nucleic acid.

10. The composition of claim 1, wherein the capture probe comprises a plurality of capture probes, and the enhancement probe comprises only one enhancement probe, wherein each of the capture probes is specific to a different variant target nucleic acid, and the enhancement probe is specific to a single reference target nucleic acid.

11. The composition of claim 1, wherein the capture probe comprises a plurality of capture probes, and the enhancement probe comprises a plurality of enhancement probes, wherein each of the capture probes is specific to a different variant target nucleic acid, and each of the enhancement probes is specific to a different reference target nucleic acid, wherein each of the reference target nucleic acids is a reference nucleic acid to one or more of the variant target nucleic acids.

12. The composition of claim 1, wherein the composition does not comprise the variant or reference target nucleic acids.

13. The composition of claim 1, wherein the composition comprises one or more of the variant and/or reference target nucleic acids.

14. The composition of claim 1, wherein the capture probe comprises at least 6 pairs of adjacent matched nucleotides with respect to the nucleic acid sequence of the strand segment of the variant target nucleic acid, and the enhancement probe comprises at least 6 pairs of adjacent matched nucleotides with respect to the nucleic acid sequence of the strand segment of the reference target nucleic acid.

15. The composition of claim 1, wherein the capture probe contains only matched nucleotides with respect to the nucleic acid sequence of the strand segment of the variant target nucleic acid, and the enhancement probe contains only matched nucleotides with respect to the nucleic acid sequence of the strand segment of the reference target nucleic acid.

16. The composition of claim 1, wherein the composition does not comprise any other oligonucleotides that are complementary to the 5′ subsequences, the intermediate subsequences, or the 3′ subsequences of the strand segments of the variant or reference target nucleic acids and does not comprise any other nucleic acids that are complementary to the capture probe or the enhancement probe.

17. A system for enriching, extracting, and detecting a variant target nucleic acid in a sample comprising the variant target nucleic acid and a reference target nucleic acid, wherein the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid, the system comprising:
(a) the composition of claim 1; and
(b) a solid, wherein the capture probe is immobilized on a surface of the solid.

18. A system for enriching, extracting, and detecting a variant target nucleic acid in a sample comprising the variant target nucleic acid and a reference target nucleic acid, wherein the variant and reference target nucleic acids each comprise a nucleic acid strand segment having a 5′ end, a 3′ end, and a nucleic acid sequence, and the nucleic acid sequences of the strand segments of the variant and reference target nucleic acids are identical except that the nucleic acid sequence of the nucleic acid strand segment of the variant target nucleic acid comprises a substitution, insertion, or deletion of 1 to 100 nucleotides, or a combination thereof, relative to the nucleic acid sequence of the nucleic acid strand segment of the reference target nucleic acid, the system comprising:
(a) the composition of claim 1; and
(b) a solid, wherein:
(i) the capture probe comprises at least one binding tag; and
(ii) the solid comprises an extraction tag that binds to the at least one binding tag.

19-37. (canceled)


SEQUENCE LISTING (45 sequences)

SEQ ID NO: 1 - DNA, 21 bp, synthetic construct
  source 1..21: mol_type = other DNA
gttgttgtgcataaaaaaaga

SEQ ID NO: 2 - DNA, 23 bp, synthetic construct
  source 1..23: mol_type = other DNA
aaagctaactggataacttacag

SEQ ID NO: 3 - DNA, 19 bp, synthetic construct
  source 1..19: mol_type = other DNA
acatgcaggaagttgttgt

SEQ ID NO: 4 - DNA, 22 bp, synthetic construct
  source 1..22: mol_type = other DNA
gttgttgtgcataaaaaaaaga

SEQ ID NO: 5 - DNA, 19 bp, synthetic construct
  source 1..19: mol_type = other DNA
cataaaaaaaagaggcctg

SEQ ID NO: 6 - DNA, 32 bp, synthetic construct
  source 1..32: mol_type = other DNA
gagatttctctgtagctagaccaaaatcaaat

SEQ ID NO: 7 - DNA, 29 bp, synthetic construct
  source 1..29: mol_type = other DNA
ctccatcgagatttcactgtagctatttt

SEQ ID NO: 8 - DNA, 22 bp, synthetic construct
  source 1..22: mol_type = other DNA
gacctcacagtaaaaataggtg

SEQ ID NO: 9 - DNA, 21 bp, synthetic construct
  source 1..21: mol_type = other DNA
atagcctcaattcttaccatc

SEQ ID NO: 10 - DNA, 33 bp, synthetic construct
  source 1..33: mol_type = other DNA
ttgtggtagttggagctgatggcgtaggctttt

SEQ ID NO: 11 - DNA, 33 bp, synthetic construct
  source 1..33: mol_type = other DNA
ttgtggtagttggagctgttggcgtaggctttt

SEQ ID NO: 12 - DNA, 33 bp, synthetic construct
  source 1..33: mol_type = other DNA
ttgtggtagttggagctgctggcgtaggctttt

SEQ ID NO: 13 - DNA, 33 bp, synthetic construct
  source 1..33: mol_type = other DNA
ttgtggtagttggagcttgtggcgtaggctttt

SEQ ID NO: 14 - DNA, 33 bp, synthetic construct
  source 1..33: mol_type = other DNA
ttgtggtagttggagctagtggcgtaggctttt

SEQ ID NO: 15 - DNA, 33 bp, synthetic construct
  source 1..33: mol_type = other DNA
ttgtggtagttggagctcgtggcgtaggctttt

SEQ ID NO: 16 - DNA, 33 bp, synthetic construct
  source 1..33: mol_type = other DNA
ttgtggtagttggagctggtgacgtaggctttt

SEQ ID NO: 17 - DNA, 31 bp, synthetic construct
  source 1..31: mol_type = other DNA
ttggagctggtggcgtaggcaagagtgaaaa

SEQ ID NO: 18 - DNA, 24 bp, synthetic construct
  source 1..24: mol_type = other DNA
ctgaaaatgactgaatataaactt

SEQ ID NO: 19 - DNA, 21 bp, synthetic construct
  source 1..21: mol_type = other DNA
tattgttggatcatattcgtc

SEQ ID NO: 20 - DNA, 56 bp, synthetic construct
  source 1..56: mol_type = other DNA
ggtcaggttaccgctgcgatcgcagagatttctctgtagctagaccaaaatcaaat

SEQ ID NO: 21 - DNA, 24 bp, synthetic construct
  source 1..24: mol_type = other DNA
tgcgatcgcagcggtaacctgacc

SEQ ID NO: 22 - DNA, 32 bp, synthetic construct
  source 1..32: mol_type = other DNA
ttggagctgatggcgtaggcaagagtgcaaaa

SEQ ID NO: 23 - DNA, 31 bp, synthetic construct
  source 1..31: mol_type = other DNA
gtggtagttggagctggtggcgtaggctttt

SEQ ID NO: 24 - DNA, 32 bp, synthetic construct
  source 1..32: mol_type = other DNA
ttggagcttgtggcgtaggcaagagtgcaaaa

SEQ ID NO: 25 - DNA, 28 bp, synthetic construct
  source 1..28: mol_type = other DNA
gagatttctctgtagctagaccaaaatc

SEQ ID NO: 26 - DNA, 43 bp, synthetic construct
  source 1..43: mol_type = other DNA
tgggtgaggtagctctaaagagacatcgatctggttttagtgg

SEQ ID NO: 27 - DNA, 25 bp, synthetic construct
  source 1..25: mol_type = other DNA
ctccatcgagatttcactgtagcta

SEQ ID NO: 28 - DNA, 43 bp, synthetic construct
  source 1..43: mol_type = other DNA
tgggtgaggtagctctaaagtgacatcgatctggttttagtgg

SEQ ID NO: 29 - DNA, 25 bp, synthetic construct
  source 1..25: mol_type = other DNA
ctccatcgagatttctctgtagcta

SEQ ID NO: 30 - DNA, 28 bp, synthetic construct
  source 1..28: mol_type = other DNA
gagatttcactgtagctagaccaaaatc

SEQ ID NO: 31 - DNA, 31 bp, synthetic construct
  source 1..31: mol_type = other DNA
gtggtagatggagctggtggcgtaggctttt

SEQ ID NO: 32 - DNA, 31 bp, synthetic construct
  source 1..31: mol_type = other DNA
gtggtagctggagctggtggcgtaggctttt

SEQ ID NO: 33 - DNA, 31 bp, synthetic construct
  source 1..31: mol_type = other DNA
gtggtaggtggagctggtggcgtaggctttt

SEQ ID NO: 34 - DNA, 32 bp, synthetic construct
  source 1..32: mol_type = other DNA
taggtgctgatggcgtatgcaagagtgcaaaa

SEQ ID NO: 35 - DNA, 30 bp, synthetic construct
  source 1..30: mol_type = other DNA
tggtagtaggtgctggtggcgtatgctttt

SEQ ID NO: 36 - DNA, 32 bp, synthetic construct
  source 1..32: mol_type = other DNA
taggtgccgatggcgaatgcaagagtgcaaaa

SEQ ID NO: 37 - DNA, 30 bp, synthetic construct
  source 1..30: mol_type = other DNA
tggtagtaggtgccggtggcgaatgctttt

SEQ ID NO: 38 - DNA, 11 bp, synthetic construct
  source 1..11: mol_type = other DNA
atttcactgta

SEQ ID NO: 39 - DNA, 11 bp, synthetic construct
  source 1..11: mol_type = other DNA
atttctctgta

SEQ ID NO: 40 - DNA, 12 bp, synthetic construct
  source 1..12: mol_type = other DNA
acgccaccagct

SEQ ID NO: 41 - DNA, 12 bp, synthetic construct
  source 1..12: mol_type = other DNA
acgccaycagct

SEQ ID NO: 42 - DNA, 12 bp, synthetic construct
  source 1..12: mol_type = other DNA
acgccacmagct

SEQ ID NO: 43 - DNA, 12 bp, synthetic construct
  source 1..12: mol_type = other DNA
acgycaccagct

SEQ ID NO: 44 - DNA, 12 bp, synthetic construct
  source 1..12: mol_type = other DNA
acgccatcagct

SEQ ID NO: 45 - DNA, 12 bp, synthetic construct
  source 1..12: mol_type = other DNA
acgccacaagct